SEQUENCE LISTING (2 sequences)

SEQ ID NO: 1 - RNA, 21 nt, Homo sapiens
cuagacugaagcuccuugagg

SEQ ID NO: 2 - RNA, 22 nt, Homo sapiens
gaauguugcucggugaaccccu

(19) United States
(12) Patent Application Publication
(10) Pub. No.: US 2014/0017230 A1
(43) Pub. Date: Jan. 16, 2014

(54) CHRONIC LYMPHOCYTIC LEUKEMIA (CLL) BIOMARKERS

(71) Applicants: HOFFMANN-LAROCHE INC., Nutley, NJ (US); GENENTECH, INC., South San Francisco, CA (US)

(72) Inventors: DAVID DORNAN, SAN MATEO, CA (US); GUILLEMETTE DUCHATEAU-NGUYEN, RIEDISHEIM (FR); TRI QUANG NGUYEN, WEIL AM RHEIN (DE); GIUSEPPE PALERMO, BASEL (CH); MARTIN WEISSER, PENZBERG (DE); RU-FANG YEH, MOUNTAIN VIEW, CA (US)

(73) Assignees: HOFFMANN-LA ROCHE INC., Nutley, NJ (US); GENENTECH, INC., South San Francisco, CA (US)

(21) Appl. No.: 13/757,600

(22) Filed: Feb. 1, 2013

Related U.S. Application Data
(63) Continuation of application No. PCT/US2011/046205, filed on Aug. 2, 2011.

(51) Int. Cl. C12Q 1/68; A61K 31/7076; A61K 31/675; A61K 39/395

(52) U.S. Cl. CPC C12Q 1/6886; A61K 39/39558; A61K 31/7076; A61K 31/675
USPC 424/133.1; 424/174.1; 506/9; 506/16

(57) ABSTRACT

The present application describes chronic lymphocytic leukemia (CLL) biomarkers. In particular, the invention concerns miRNA151 3p, miRNA409 3p, PTK2, and/or PI3K as biomarkers for patient selection in CLL, as well as methods of therapeutic treatment, articles of manufacture and methods for making them, diagnostic kits, and methods of advertising related thereto.

### CROSS REFERENCE TO RELATED APPLICATIONS

This application is a continuation of International Application No. PCT/US2011/046205 having an international filing date of Aug. 2, 2011, which claims benefit under 35 USC §119 to U.S. Provisional Application No. 61/370,403, filed Aug. 3, 2010 and to U.S. Provisional Application No. 61/440,162, filed Feb. 7, 2011, the contents of which are incorporated by reference in its entirety.

### SEQUENCE LISTING

The instant application contains a Sequence Listing submitted via EFS-Web and is hereby incorporated by reference in its entirety. Said ASCII copy, created on Jan. 31, 2013, is named P4456R1C1SeqList.txt, and is 501 bytes in size.

### FIELD OF THE INVENTION

The present invention concerns chronic lymphocytic leukemia (CLL) biomarkers. In particular, the invention concerns miRNA151 3p, miRNA409 3p, PTK2, and PI3K, as biomarkers for patient selection in CLL, as well as methods of therapeutic treatment, articles of manufacture and methods for making them, diagnostic kits, and methods of advertising related thereto.

### BACKGROUND

Chronic lymphocytic leukemia (CLL) is the most common form of adult leukemia in the western world. Akin to many other B-cells malignancies, CLL is characterized by the expression of the CD20 surface antigen and can thereby be targeted by anti-CD20 therapy. Rituximab, a monoclonal chimeric anti-CD20 antibody has demonstrated significant benefit for patients with follicular NHL (Marcus et al., J Clin Oncol. 26:4579-4586 (2008); Hiddemann et al., Blood 106:3725-3732 (2005)) and diffuse large B-cell lymphoma (DLBCL) (Coiffier et al., N. Engl J. Med. 346:235-242 (2002); Feugier et al., J Clin Oncol. 23(18):4117-4126 (2005)) with respect to Progression Free Survival (PFS) and Overall Survival (OS). In addition, chemoimmunotherapy with rituximab has also shown to prolong PFS in CLL in untreated and relapsed/refractory patients as compared to chemotherapy alone (Hallek et al., Blood, ASH Annual Meeting Abstract, 112: 325 (2008); Robak et al., Blood, ASH Annual Meeting Abstracts, 112:lba-1 (2008)) and more recently a prolongation of overall survival (Hallek et al., Blood, ASH Annual Meeting Abstracts, 114: Abstract 535 (2009)).

The mechanisms of action by which rituximab clears B-cells includes antibody dependent cellular cytotoxicity (ADCC) (Golay et al., Blood 95(12):3900-3908 (2000)), complement dependent cytotoxicity (CDC) (Golay et al., Blood 95(12):3900-3908 (2000); Harjunpaa et al., Scand J Immunol. 51(6):634-641(2000)) and direct proapoptotic effects (Hofmeister et al., Blood Cells Mol Dis. 6(2):133-143 (2000)).

Despite the knowledge of mechanism of action of the anti-CD20 antibody rituximab, there is still lack of knowledge regarding disease or host related factors that would predict response or resistance to CD20 antibody-based therapy. In CLL numerous factors have been published that have demonstrated to influence the prognosis of the disease. These factors include cytogenetic and molecular aberrations, mutational status of the IgVH locus, ZAP70 and CD38 expression and many more (reviewed in Moreno and Monserrat Blood Reviews 22:211-219 (2008)).

However, none of these markers has to date shown true predictive significance for anti-CD20 antibody based therapy in CLL. In addition, Fcγ receptor polymorphisms that have been linked to the mechanism of action in other NHL entities (Cartron et al., Blood 99(3):754-758 (2002); Weng and Levy, J Clin Oncol. 21(21):3940-7 (2003)) have not been shown to influence the outcome of anti-CD20 based therapy in CLL (Farag et al., Blood 103:1472-1474 (2004); Dornan et al., Blood (ASH Annual Meeting Abstracts) 114: 2338 (2009)).

The aim of this study was to discover predictive biomarkers in a large cohort of patients treated within a controlled randomized trial treated with either standard chemotherapy (fludarabine/cyclophosphamide; FC) or FC plus rituximab (R-FC) and correlate the genomic data to clinical outcome.

MicroRNAs (miRNAs) are short (17-27 nucleotide) non-coding RNAs involved in post-transcriptional regulation of gene expression in multicellular organisms by affecting both the stability and translation of mRNAs. MiRNAs can bind to target mRNAs and either inhibit translation or promote degradation thereof, thereby affecting biologic processes. miRNA151 3p is a microRNA found on chromosome 8, which is intronic of PTK2. Publications regarding miRNA151 3p include: Fulci et al. Genes, Chromosomes & Cancer 48 (12): 1069-1082 (2009); Agirre et al. Mol. Cancer Res. 6 (12): 1830-1840 (2008); Ding et al. Nat. Cell Biol. 12(4): 390-399 (2010); Kawahara et al. EMBO 8(8): 763-769 (2007).

Visone et al. Blood 114(18): 3872-3879 (2009) concerns karyotype-specific miRNA signature in CLL.

### SUMMARY OF THE INVENTION

The invention herein concerns the identification of biomarkers miRNA151 3p, miRNA409 3p, PTK2, and PI3K as predicting response to therapy in CLL.

According to a first embodiment, the invention concerns a method for treating a patient with chronic lymphocytic leukemia (CLL) comprising administering a therapeutically effective amount of a CLL medicament to the patient if the patient has been found to have an elevated amount of one or more biomarker selected from miRNA151 3p, miRNA409 3p, and PTK2. Examples of such CLL medicament include:

CLL medicaments which induce FAK signaling and/or homotypic adhesion;

B-cell antagonists, such as CD20 antibodies, e.g. humanized, human, or chimeric anti-CD20 antibodies

Type I anti-CD20 antibodies

Type II anti-CD20 antibodies

Anti-CD20 antibodies such as rituximab, ofatumumab, GA101, SBI-087, veltuzumab, and AME-133.

In another embodiment, the invention provides a method for treating a patient with chronic lymphocytic leukemia (CLL) comprising administering to the patient a therapeutically effective amount of a combination of rituximab, fludarabine and cyclophosphamide, if the patient has been found to have an elevated amount of one or more biomarker selected from miRNA151 3p, miRNA409 3p, and PTK2.

In addition, the invention concerns a method for treating a patient with chronic lymphocytic leukemia (CLL) comprising administering to the patient a therapeutically effective amount of CLL medicament other than rituximab, if the patient has been found to have a reduced amount of one or more biomarker selected from miRNA151 3p, miRNA409 3p, and PTK2.

The invention also relates to a method for selecting a therapy for a patient with chronic lymphocytic leukemia (CLL) comprising determining expression of a biomarker selected from miRNA151 3p, miRNA409 3p, PTK2, and PI3K, in a sample from the patient, and selecting a CLL medicament based on the level of expression of the biomarker. In one embodiment, the patient is selected for treatment with a CLL medicament (e.g. one which induces FAK signaling or homotypic adhesion, or which is a B-cell antagonist such as a CD20 antibody) if the cancer sample expresses the biomarker at an elevated level. In another embodiment, the patient is selected for treatment with a CLL medicament other than rituximab if the cancer sample expresses the biomarker at a reduced level.

The invention also provides a diagnostic kit comprising one or more reagents for determining expression of a biomarker selected from miRNA151 3p, miRNA409 3p, PTK2, and PI3K, in a sample from a CLL patient.

The invention also concerns a article of manufacture comprising, packaged together, a CLL medicament in a pharmaceutically acceptable carrier and a package insert indicating that the CLL medicament is for treating a patient with chronic lymphocytic leukemia (CLL) based on expression of one or more biomarker selected from miRNA151 3p, miRNA409 3p, PTK2, and PI3K.

In a related aspect, the invention concerns a method for manufacturing an article of manufacture comprising combining in a package a pharmaceutical composition comprising a CLL medicament and a package insert indicating that the pharmaceutical composition is for treating a patient with chronic lymphocytic leukemia (CLL) based on expression of one or more biomarker selected from miRNA151 3p, miRNA409 3p, PTK2, and PI3K.

Moreover, the invention concerns a method for advertising a CLL medicament comprising promoting, to a target audience, the use of the CLL medicament for treating a patient with chronic lymphocytic leukemia (CLL) based on expression of one or more biomarker selected from miRNA151 3p, miRNA409 3p, PTK2, and PI3K.

In another aspect, the invention concerns a method for treating a patient with chronic lymphocytic leukemia (CLL) comprising administering a therapeutically effective amount of a CLL medicament to the patient if the patient has been found to have reduced PI3K biomarker.

Moreover, the invention, in one aspect provides a method for treating a patient with chronic lymphocytic leukemia (CLL) comprising administering to the patient a therapeutically effective amount of a combination of rituximab, fludarabine and cyclophosphamide, if the patient has been found to have reduced PI3K biomarker.

In addition, a method for treating a patient with chronic lymphocytic leukemia (CLL) is provided comprising administering to the patient a therapeutically effective amount of CLL medicament other than rituximab, if the patient has been found to have elevated PI3K biomarker.

### BRIEF DESCRIPTION OF THE FIGURES

FIG. 1A depicts progression free survival (PFS) with respect to miRNA151 3p expression (array based) and therapy.

FIG. 1B depicts PFS with respect to miRNA151 3p expression (qRT-PCR based) and therapy.

FIG. 2A depicts PFS with respect to miRNA409 3p expression (array based) and therapy.

FIG. 2B depicts PFS with respect to miRNA409 3p expression (qRT-PCR based) and therapy.

FIG. 3 depicts AFFYMETRIX® Exon 1.0 ST PFS with respect to treatment and PTK2 expression.

FIG. 4 depicts AFFYMETRIX® U133+2 PFS with respect to treatment and PTK2 expression.

FIG. 5 depicts qRT-PCR: PFS with respect to treatment and PTK2 expression.

FIG. 6 depicts targeting of 3′UTRs by miRNA151 3p. HeLa cells were transfected with miRNA151 3p or NTC as described in methods. Repression of each construct by miRNA151 3p was normalized to NTC. Data are derived from three biological replicates. Statistical significance was determined by a 2-sided Student's t-test. **P<0.01, *P<0.05.

FIG. 7 depicts PFS in patients stratified by treatment and PIK3R3 expression (high: >=median=4; low: <4).

FIGS. 8A-8C depict outcome association for PIK3R3 expression: FCR vs. FC treatment effect of PIK3R3 expression subgroups (FIG. 8A); prognostic effect of PIK3R3 expression in FC arm (FIG. 8B); and effect of PIK3R3 expression in FCR arm (FIG. 8C). Marker cutoff refers to PIK3R3 expression at the specified quartile or as a continuous variable.

### DETAILED DESCRIPTION OF EMBODIMENTS OF THE INVENTION

### I. Definitions

“Chronic lymphocytic leukemia” or “CLL” refers to cancer of white blood cells (lymphocytes). Examples of CLL herein include, “first line” or “untreated” CLL (i.e. where the CLL patient has received no prior therapy for treating the CLL), “previously treated CLL” (where the CLL patient has received prior therapy for the CLL), “refractory” CLL (where the patient is refractory to therapy for CLL), “relapsed CLL” (where the patient has relapsed following prior therapy for the CLL).

Herein, a “patient” is a human patient. The patient may be a “CLL patient,” i.e. one who is suffering or at risk for suffering from one or more symptoms of CLL. Moreover, the patient may be a previously treated CLL patient.

For the purposes herein, a “previously treated” CLL patient has received prior CLL therapy, such therapy including chlorambucil (with or without prednisone/prednisolone), fludarabine (or other nucleoside analog), and/or an alkyaltor-containing combination regimen. Such previously treated CLL patients are optionally sensitive or reflactory to prior aklylating agents, but are preferably sensitive to fludarabine (e.g. achieving a response that lasted 6 months or more).

“Refractory” CLL progresses even though an anti-tumor agent, such as a chemotherapeutic agent, is being administered to the CLL patient. An example of a refractory cancer is one which is refractory to any one or more of: nucleoside analogue (e.g. fludarabine), cyclophosphamide; fludarabine and cyclophosphamide (FC); chlorambucil; prednisone or prednisolone; akylator-containing combination therapy, including cyclophosphamide, vincristine, prednisolone (CHOP), or cyclophosphamide, vincristine, prednisolone (CVP); alemtuzumab (Campath); etc.

A “CLL medicament” is a drug effective for treating CLL. Examples of CLL medicaments include the chemotherapeutic agents and chemotherapy regimens noted below; B cell antagonists, such as CD20 antibodies (e.g. rituximab or ofatumumab etc), CD22 antibodies, and CD79b antibodies; intravenous immune globulin; CD52 antibodies (e.g. alemtuzumab); alkylating agents (e.g. chlorambucil, bendamustine, or cyclophosphamide); nucleoside analogues or antimetabolites (e.g. fludarabine), fludarabine and cyclophosphamide (FC); prednisone or prednisolone; akylator-containing combination therapy, including cyclophosphamide, vincristine, prednisolone (CHOP), or cyclophosphamide, vincristine, prednisolone (CVP); etc. In one embodiment, the CLL medicament induces FAK signaling and/or homotypic aggregation.

“FAK signaling” refers to the upregulation or activation of FAK (e.g. via phosphorylation of Tyr³⁹⁷ of FAK) including activation of downstream signaling molecules such as Src kinase, growth factor receptor binding protein 2 adaptor protein (Grb2), and/or Ras/mitogen-activated protein kinase pathway due to FAK activation. Methods for identifying CLL medicaments or B-cell antagonists (e.g. CD20 antibodies) which induce FAK signaling are described in Altomonte et al. J. Cell. Physiol 200: 272-274 (2004).

“Homotypic adhesin” or “homotypic aggregation” refers to interaction and attachment of the same cells to each other, which may lead to programmed cell death. Methods for identifying CLL medicaments or B-cell antagonists (e.g. CD20 antibodies) which induce homotypic aggregation can be evaluated as described in Ivanov et al. J. Clin. Invest. 119(8): 2143-2159 (2009) or Altomonte et al. J. Cell. Physiol 200: 272-274 (2004).

The term “biomarker” or “marker” as used herein refers generally to a molecule, including a gene, mRNA, protein, carbohydrate structure, or glycolipid, the expression of which in or on a tissue or cell or secreted can be detected by known methods (or methods disclosed herein) and is predictive or can be used to predict (or aid prediction) for a cell, tissue, or patient's responsiveness to treatment regimes. The biomarkers of particular interest herein are PTK2, miRNA151 3p, and miRNA409 3p. “Protein-tyrosine kinase 2” or “PTK2” when used herein refers to human protein tyrosine kinase 2 (PTK2) also known as focal adhesion kinase 1 (FADK 1). For the purposes herein, “PTK2” refers to DNA or mRNA encoding PTK2, as well as PTK2 protein encoded, including fragments or portions of any of these which facilitate detection of PTK2 in a sample. PTK2 sequences are publicly available at:

http://www.ncbi.nlm.nih.gov/sites/entrez?db=gene&cmd=Retrieve&dopt=Graphics&list_uids=5747, or

http://www.uniprot.org/uniprot/Q05397.

PTK2 is also known as: FAK; FADK; FAK1; FRNK; pp125FAK; PTK2. PTK2 gene encodes a cytoplasmic protein tyrosine kinase which is found concentrated in the focal adhesions that form between cells growing in the presence of extracellular matrix constituents. The encoded protein is a member of the focal adhesion kinase (FAK) subfamily of protein tyrosine kinases but lacks significant sequence similarity to kinases from other subfamilies. Activation of this gene may be an important early step in cell growth and intracellular signal transduction pathways triggered in response to certain neural peptides or to cell interactions with the extracellular matrix. At least four transcript variants encoding four different isoforms have been found for this gene, but the full-length natures of only two of them have been determined. The term “PTK2” herein includes each of the isoforms thereof, including isoforms 1, 2, 3, and 4 (Q05397-1, -2, -3, and -4 respectively). PTK2 DNA, mRNA, and/or protein can be evaluated according to the present invention.

“Phosphoinositide 3-kinase” and “PI3K” refer to human phosphoinositide 3-kinase, including subunits thereof. PI3Ks are lipid kinases capable of phosphyorylating the 3′OH of the inositol ring of phosphoinositides. PI3K herein includes Class I (including Class IA and Class 1B), Class II, and Class III PI3Ks. In one embodiment, the PI3K is a Class I PI3K, and, optionally, the PI3K comprises a regulatory subunit thereof, such as regulatory subunit 3 (PIK3R3). For the purposes herein, PI3K (and synonyms) refer to DNA or mRNA encoding PI3K, or PI3K protein (including a subunit of PI3K), as well as fragments or portions of any of these which facilitate detection of PI3K in a sample. The PI3K DNA, mRNA, and/or protein, as well as PI3K activation, can be evaluated according to the present invention. Thus, “reduced PI3K biomarker” refers to reduced amount and/or activity, of P13K biomarker, and “elevated PI3K biomarker” refers increased amount and/or activity, of P13K biomarker.

“Phosphoinositide-3-kinase, regulatory subunit 3 (gamma),” “PIK3R3,” and “p55-gamma” refer to human regulatory subunit 3 of PI3K capable of interacting with PI3K catalytic subunits (110 kDa). See, for example, Dey et al. Gene 209(1-2):175-83 (1998), Ingham et al. J. Biol. Chem. 276(15): 12257-65 (2001), and UniProtKB/Swiss-Prot: P55G HUMAN.Q92569. PIK3R3 isoforms are expressly included in this definition. For the purposes herein, PIK3R3 (and synonyms) refer to DNA or mRNA encoding PIK3K3, or PIK3R3 protein, as well as fragments or portions of any of these which facilitate detection of PIK3R3 in a sample. PIK3R3 DNA, mRNA, and/or protein, and/or PIK3R3 activation (or activation of PI3K comprising PIK3R3), can be evaluated according to the present invention.

A “microRNA” or “miRNA” is a short (generally about 15-30 nucleotide) non-coding RNA that is involved in post-transcriptional regulation of gene expression in multicellular organisms by affecting both the stability and translation of mRNAs.

“miRNA151 3p” when used herein refers to a miRNA comprising the sequence: CUAGACUGAAGCUCCUUGAGG (SEQ ID NO: 1). See also: http://www.ncbi.nlm.nih.gov/gene/442893 and http://www.mirbase.org/cgi-bin/mirna_entry.pl?acc=MI0000809. miRNA151 3p is intronic to and co-expressed with PTK. For the purposes herein, the term includes DNA or mRNA including fragments or portions thereof which facilitate detection of miRNA151 3p in a sample.

“miRNA409 3p” used herein refers to RNA comprising the sequence: GAAUGUUGCUCGGUGAACCCCU (SEQ ID NO: 2). See, also: http://www.ncbi.nlm.nih.gov/gene/574413 or http://www.mirbase.org/cgi-bin/mirna_entry.pl?acc=MI0001735. For the purposes herein, the term includes DNA or mRNA including fragments or portions thereof which facilitate detection of miRNA409 3p in a sample.

By “patient sample” is meant a collection of similar cells obtained from a CLL patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may contain compounds which are not naturally intermixed with the tissue in nature such as preservatives, anticoagulants, buffers, fixatives, nutrients, antibiotics, or the like. Examples of tumor samples herein include, but are not limited to, tumor biopsies, circulating tumor cells, serum or plasma, Peripheral Blood Mononuclear Cells (PBMCs), circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples. In one embodiment the sample comprises Peripheral Blood Mononuclear Cells (PBMCs), including CD19-enriched PBMCs.

An “effective response” of a patient or a patient's “responsiveness” to treatment with a medicament and similar wording refers to the clinical or therapeutic benefit imparted to a patient at risk for, or suffering from, CLL upon administration of the CLL medicament. Such benefit includes any one or more of: extending survival (including overall survival and progression free survival); resulting in an objective response (including a complete response or a partial response); improving signs or symptoms of CLL, etc. In one embodiment, the biomarker is used to identify the patient who is expected to have greater progression free survival (PFS) when treated with a medicament, relative to a patient who does not express the biomarker at the same level. The incidence of biomarker(s) herein effectively predicts, or predicts with high sensitivity, such effective response.

“Survival” refers to the patient remaining alive, and includes overall survival as well as progression free survival.

“Overall survival” refers to the patient remaining alive for a defined period of time, such as 1 year, 5 years, etc from the time of diagnosis or treatment.

“Progression free survival” refers to the patient remaining alive, without the cancer progressing or getting worse.

By “extending survival” is meant increasing overall or progression free survival in a treated patient relative to an untreated patient (i.e. relative to a patient not treated with the medicament), or relative to a patient who does not express a biomarker at the designated level, and/or relative to a patient treated with an approved anti-tumor agent (such as chemotherapy regimen of fludarabine and cyclophosphamide, FC).

An “objective response” refers to a measurable response, including complete response (CR) or partial response (PR).

By “complete response” or “CR” is intended the disappearance of all signs of cancer in response to treatment. This does not always mean the cancer has been cured.

“Partial response” or “PR” refers to a decrease in the size of one or more tumors or lesions, or in the extent of cancer in the body, in response to treatment.

The “amount” or “level” of a biomarker associated with an increased clinical benefit to a CLL patient is a detectable level in a biological sample. These can be measured by methods known to the expert skilled in the art and also disclosed by this invention. The expression level or amount of biomarker assessed can be used to determine the response to the treatment.

The terms “level of expression” or “expression level” in general are used interchangeably and generally refer to the amount of a polynucleotide, mRNA, or an amino acid product or protein in a biological sample. “Expression” generally refers to the process by which gene-encoded information is converted into the structures present and operating in the cell. Therefore, according to the invention “expression” of a gene may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, miRNAs).

An “elevated” or “high” amount or level of a biomarker refers to an amount that is equal to or greater than the median amount of the biomarker in a patient population, e.g. in a population of patients with CLL, or in a subpopulation of CLL patients (e.g. previously treated CLL patients). For example, the amount may be in a percentile range from 50% to about 100%, e.g. from about 75-100%.

A “reduced” or “low” amount or level of a biomarker refers to an an amount that is lower than the median amount of the biomarker in a patient population. For example, the amount may be in a percentile range from 0% to about 49%, e.g. from about 0-25%.

The phrase “based on expression of” when used herein means that information about expression level of the one or more biomarkers herein is used to inform a treatment decision, information provided on a package insert, or marketing/promotional guidance etc. In the case of elevated expression of the biomarker, patients may be treated with a CLL medicament such as a B-cell antagonist, e.g. CD20 antibody, such as rituximab. In the case of reduced level of expression of the biomarker, patients may be treated with a CLL medicament other than rituximab (or other than an anti-CD20 antibody).

A “B-cell surface marker” or “B-cell surface antigen” herein is an antigen expressed on the surface of a B cell that can be targeted with an antagonist that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers (for descriptions, see The Leukocyte Antigen Facts Book, 2^{nd} Edition. 1997, ed. Barclay et al. Academic Press, Harcourt Brace & Co., New York). Other B-cell surface markers include RP105, FcRH2, B-cell CR2, CCR6, P2X5, HLA-DOB, CXCR5, FCER2, BR3, BAFF, BLyS, Btig, NAG14, SLGC16270, FcRH1, IRTA2, ATWD578, FcRH3, IRTA1, FcRH6, BCMA, and 239287. In one embodiment, the B-cell surface marker is CD20, CD22, or CD79b.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35”. The CD20 antigen is described in Clark et al., Proc. Natl. Acad. Sci. (USA), 82:1766 (1985), for example.

An “B-cell antagonist” is a molecule that, upon binding to a B-cell surface marker or B-cell specific survival or proliferation factor, destroys or depletes B cells in a mammal and/or interferes with B-cell survival and/or one or more B-cell functions, e.g. by reducing or preventing a humoral response elicited by the B cell. The antagonist preferably is able to deplete B cells (i.e. reduce circulating B-cell levels) in a mammal treated therewith. Such depletion may be achieved via various mechanisms such as ADCC and/or CDC, inhibition of B-cell proliferation or direct lysis of B-cells and/or induction of B-cell death (e.g. via apoptosis). Antagonists can be screened by various methods known in the art for apoptosis and other measurements for the depletion, and retardation or stopping of proliferation and growth of B cells or survival of B cells.

An “antibody that binds to a B-cell surface marker” or “antibody to a B-cell surface marker” is a molecule that, upon binding to a B-cell surface marker, destroys or depletes B cells in a mammal and/or interferes with one or more B-cell functions, e.g. by reducing or preventing a humoral response elicited by the B cell. The antibody preferably is able to deplete B cells (i.e. reduce circulating B-cell levels) in a mammal treated therewith. Such depletion may be achieved via various mechanisms such antibody-dependent cell-mediated cytotoxicity (ADCC) and/or complement-dependent cytotoxicity (CDC), inhibition of B-cell proliferation, and/or induction of B-cell death (e.g. via apoptosis). In one embodiment, the antibody is an antibody which binds human CD20.

Examples of anti-CD20 antibodies include: chimeric anti-CD20 antibodies such as rituximab (see below); human anti-CD20 antibodies such as ofatumumab (sold by Genmab, Denmark; see, also, Glennie and van de Winkel, Drug Discovery Today 8: 503-510 (2003); Cragg et al., Blood 101: 1045-1052 (2003); WO 2004/035607; and WO 2005/103081); humanized anti-CD20 antibodies such as humanized 2H7 (see below) or veltuzumab (hA20) (Goldenberg et al. Blood 113(5): 1062-1070 (2009)); glycosylation variant antibodies, including glycosylation variants with bisected, afucosylated Fc region-carbohydrates such as GA101 (see below); Small Modular ImmunoPharmaceutical (SMIP) or single-chain antibodies such as SBI-087; the yttrium-[90]-labelled 2B8 murine antibody designated “Y2B8” or “Ibritumomab Tiuxetan” (ZEVALIN®) commercially available from Biogen Idec, Inc. (e.g., U.S. Pat. No. 5,736,137; 2B8 deposited with ATCC under accession no. HB11388 on Jun. 22, 1993); murine IgG2a “B1,” also called “Tositumomab,” optionally labelled with ¹³¹I to generate the “131I-B1” or “iodine I131 tositumomab” antibody (BEXXAR™) commercially available from Corixa (see, also, e.g., U.S. Pat. No. 5,595,721); murine monoclonal antibody “1F5” (e.g., Press et al. Blood 69(2):584-591 (1987) and variants thereof including “framework patched” or humanized 1F5 (e.g., WO 2003/002607, Leung, S.; ATCC deposit HB-96450); the antibodies having complex N-glycoside-linked sugar chains bound to the Fc region described in US 2004/0093621 (Shitara et al.); a chimerized or humanized monoclonal antibody having a high binding affinity to an extracellular epitope of a CD20 antigen described in WO 2006/106959 (Numazaki et al., Biomedics Inc.); monoclonal antibodies and antigen-binding fragments binding to CD20 (e.g., WO 2005/000901, Tedder et al.) such as HB20-3, HB20-4, HB20-25, and MB20-11; single-chain proteins binding to CD20 including, but not limited to, TRU-015 (e.g., US 2005/0186216 (Ledbetter and Hayden-Ledbetter); US 2005/0202534 (Hayden-Ledbetter and Ledbetter); US 2005/0202028 (Hayden-Ledbetter and Ledbetter); US 2005/136049 (Ledbetter et al.); US 2005/0202023 (Hayden-Ledbetter and Ledbetter)—Trubion Pharm Inc.); CD20-binding molecules such as the AME series of antibodies, e.g., AME-133 antibodies as set forth, for example, in WO 2004/103404; US 2005/0025764; and US 2006/0251652 (Watkins et al., Applied Molecular Evolution, Inc.) and the anti-CD20 antibodies with Fc mutations as set forth, for example, in WO 2005/070963 (Allan et al., Applied Molecular Evolution, Inc.); CD20-binding molecules such as those described in WO 2005/016969 and US 2005/0069545 (Carr et al.); bispecific antibodies as set forth, for example, in WO 2005/014618 (Chang et al.); fully human antibodies against CD20 as described, e.g., in WO 2006/130458; Gazit et al., Amgen/AstraZeneca); antibodies against CD20 as described, for example, in WO 2006/126069 (Morawala, Avestha Gengraine Technologies Pvt Ltd.); A20 antibody or variants thereof such as chimeric or humanized A20 antibody (cA20, hA20, respectively) and IMMUN-106 (e.g., US 2003/0219433, Immunomedics); and monoclonal antibodies L27, G28-2, 93-1B3, B-C1 or NU-B2 available from the International Leukocyte Typing Workshop (e.g., Valentine et al., In: Leukocyte Typing III (McMichael, Ed., p. 440, Oxford University Press (1987)).

A “Type I” CD20 antibody mediates cell death via potent complement-dependent cytotoxicity (CDC) and antibody-dependent cellular cytotoxicity (ADCC). Examples of Type I anti-CD20 antibodies include rituximab, veltuzumab, ocrelizumab, ofatumumab, and AME-133.

A “Type II” CD20 antibody initiates target cell death via caspase-independent apoptosis with concomitant phosphatidylserine exposure and exhibits stronger homotypic adhesion and ADCC than a Type I anti-CD20 antibody. Type II antibodies do not localize CD20 into lipid rafts. Examples of Type II anti-CD20 antibodies include tositumomab (B1), 11B8, AT80 and humanized B-Ly1 antibodies.

“Rituximab” is a chimeric IgG1 anti-CD20 antibody that binds to the CD20 antigen on B cells and depletes B cells in vivo. The amino acid sequences of its heavy and light chains are disclosed in U.S. Pat. No. 7,381,560, expressly incorporated herein by reference. Rituximab is commercially available from Genentech in the United States under the trademark RITUXAN®, and is commercially available in other countries from Roche where it is sold under the trademark MABTHERA®. Nucleic acid encoding rituximab has been deposited on Nov. 10, 1992 with the ATCC under deposit no. 69119. Rituximab's biological activity is understood to involve signaling in target cells on CD20 binding leading to growth inhibition and (nonclassic) apoptosis (referred to as “direct cell death”), complement-dependent cytotoxicity (CDC), and antibody-dependent cellular cytotoxicity (ADCC) mediated by cells displaying Fcγ receptors (FcγRs), such as FcγRIIIa-expressing NK cells and macrophages.

The terms “humanized B-Ly1 antibody” and “GA101” herein refer to humanized IgG1 B-Ly1 antibody as disclosed in WO2005/044859 and WO2007/031875, which describe humanized variants of murine monoclonal anti-CD20 antibody B-Ly1. Furthermore the humanized B-Ly1 antibody is preferably glycoengineered (GE) in the Fc region according to the procedures described in WO2005/044859, WO2004/065540, WO2007/031875, Umana et al. Nature Biotechnol. 17 (1999) 176-180 (1999), and WO1999/54342. The afucosylated glyco-engineered humanized B-Ly1 (B-HH6-B-KV1 GE) is preferred in one embodiment of the invention. Such glycoengineered humanized B-Ly1 antibodies have an altered pattern of glycosylation in the Fc region, preferably having a reduced level of fucose residues. Preferably the amount of fucose is 60% or less of the total amount of oligosaccharides at Asn297 (in one embodiment the amount of fucose is between 40% and 60%, in another embodiment the amount of fucose is 50% or less, and in still another embodiment the amount of fucose is 30% or less). Furthermore the oligosaccharides of the Fc region are preferably bisected. These glycoengineered humanized B-Ly1 antibodies have an increased ADCC.

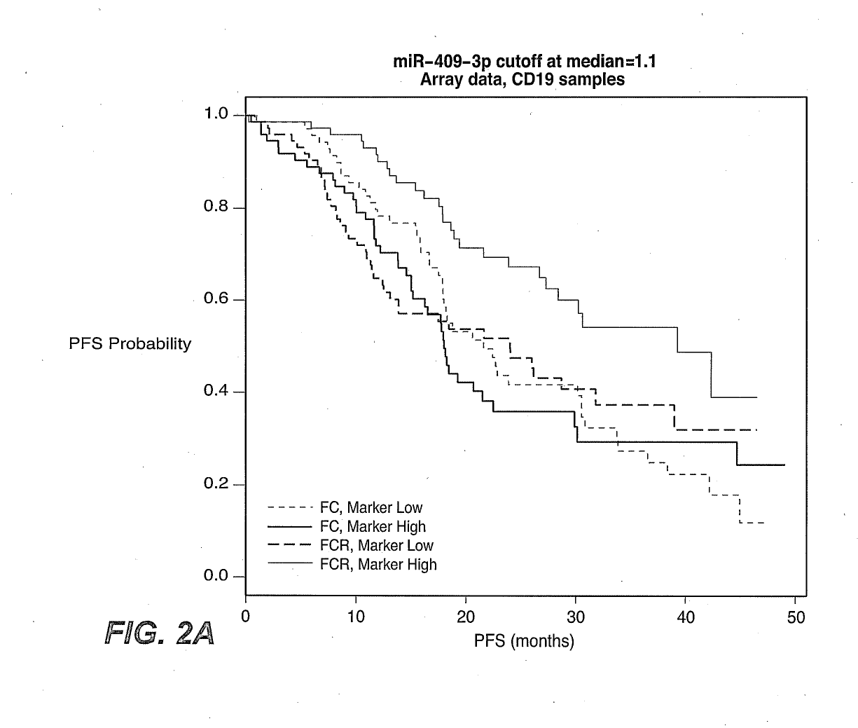

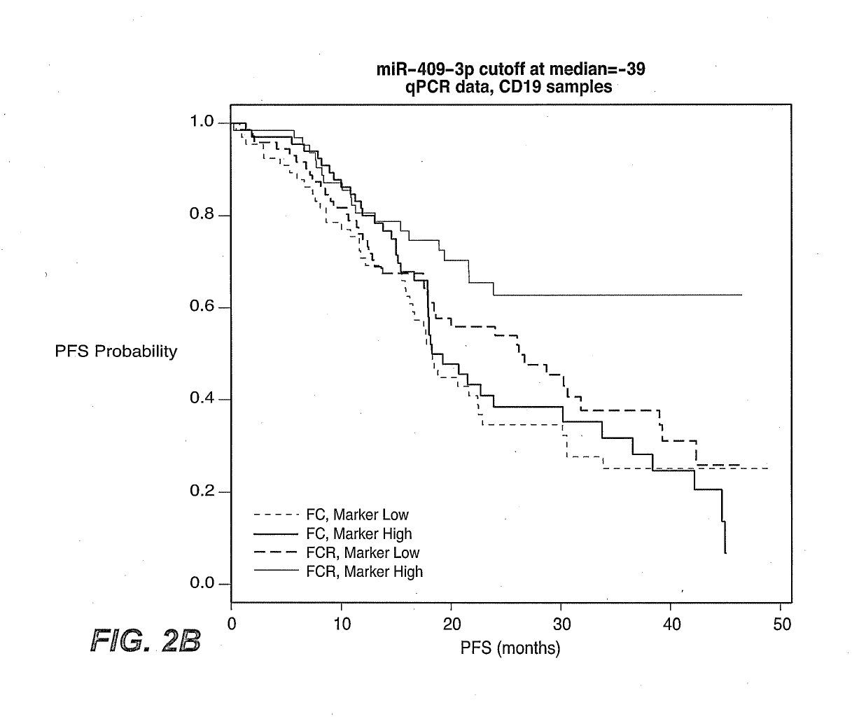

Purely for the purposes herein and unless indicated otherwise, “2H7” or “2H7 antibody” refers to a humanized anti-CD20 antibody comprising the variable domain sequences described in US 2006/0034835 and WO 2004/056312 (both Lowman et al.); US 2006/0188495 (Barron et al.); and US 2006/0246004 (Adams et al.). Briefly, humanization of the murine anti-human CD20 antibody, 2H7 (also referred to herein as m2H7, m for murine), was carried out in a series of site-directed mutagenesis steps. The murine 2H7 antibody variable region sequences and the chimeric 2H7 with the mouse V and human C have been described, e.g., in U.S. Pat. Nos. 5,846,818 and 6,204,023. The CDR residues of 2H7 were identified by comparing the amino acid sequence of the murine 2H7 variable domains (disclosed in U.S. Pat. No. 5,846,818) with the sequences of known antibodies (Kabat et al., Sequences of Proteins of Immunological Interest, Ed. 5 (Public Health Service, National Institutes of Health, Bethesda, Md., 1991)). The CDR regions for the light and heavy chains were defined based on sequence hypervariability (Kabat et al., supra). Using synthetic oligonucleotides, site-directed mutagenesis (Kunkel, Proc. Natl. Acad. Sci. (USA), 82:488-492 (1985)) was used to introduce all six of the murine 2H7 CDR regions into a complete human Fab framework corresponding to a consensus sequence V_{κ}I,V_{H}III (V_{L} kappa subgroup I, V_{H} subgroup III) contained on plasmid pVX4 (see FIG. 2 in WO 2004/056312). Further modifications of the V regions (CDR and/or FR) were made in the phagemid pVX4 by site-directed mutagenesis. Plasmids for expression of full-length IgG's were constructed by subcloning the V_{L} and V_{H} domains of chimeric 2H7 Fab as well as humanized Fab versions 2 to 6 into previously described pRK vectors for mammalian cell expression (Gorman et al., DNA Prot. Eng. Tech., 2:3-10 (1990)). Ocrelizumab is an example of a humanized 2H7 antibody herein.

The technique of “polymerase chain reaction” or “PCR” as used herein generally refers to a procedure wherein minute amounts of a specific piece of nucleic acid, RNA and/or DNA, are amplified as described in U.S. Pat. No. 4,683,195 issued 28 Jul. 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263 (1987); Erlich, ed., PCR Technology, (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Quantitative real time polymerase chain reaction” or “qRT-PCR” refers to a form of PCR wherein the amount of PCR product is measured at each step in a PCR reaction. This technique has been described in various publications including Cronin et al., Am. J. Pathol. 164(1):35-42 (2004); and Ma et al., Cancer Cell 5:607-616 (2004).

The term “microarray” refers to an ordered arrangement of hybridizable array elements, preferably polynucleotide probes, on a substrate.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 &gr; g/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin, and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovovin; novantrone; edatrexate; daunomycin; aminopterin; ibandronate; topoisomerase inhibitor RFS 2000; difluorometlhylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Herein, chemotherapeutic agents include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releasing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

Specific examples of chemotherapeutic agents or chemotherapy regimens herein include: alkylating agents (e.g. chlorambucil, bendamustine, or cyclophosphamide); nucleoside analogues or antimetabolites (e.g. fludarabine), fludarabine and cyclophosphamide (FC);

prednisone or prednisolone; akylator-containing combination therapy, including cyclophosphamide, vincristine, prednisolone (CHOP), or cyclophosphamide, vincristine, prednisolone (CVP), etc.

A “target audience” is a group of people or an institution to whom or to which a particular medicament is being promoted or intended to be promoted, as by marketing or advertising, especially for particular uses, treatments, or indications, such as individual patients, patient populations, readers of newspapers, medical literature, and magazines, television or internet viewers, radio or internet listeners, physicians, drug companies, etc.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein. In one embodiment the antibody binds to the same epitope as rituximab, ofatumumab, GA101, SBI-087, veltuzumab, or AME-133.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987)). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991)) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008)). Unless otherwise indicated, HVR residues and other residues in the variable domain (e.g., FR residues) are numbered herein according to Kabat et al., supra. In one embodiment, the CD20 antibody herein comprises the HVRs of rituximab, ofatumumab, GA101, SBI-087, veltuzumab, or AME-133.

An “immunoconjugate” is an antibody conjugated to one or more heterologous molecule(s), including but not limited to a cytotoxic agent.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical formulation” refers to a sterile preparation that is in such form as to permit the biological activity of the medicament to be effective, and which contains no additional components that are unacceptably toxic to a subject to which the formulation would be administered.

A “sterile” formulation is aseptic or free from all living microorganisms and their spores.

A “kit” is any manufacture (e.g a package or container) comprising at least one reagent, e.g., a medicament for treatment of CLL, or a probe for specifically detecting a biomarker gene or protein of the invention. The manufacture is preferably promoted, distributed, or sold as a unit for performing the methods of the present invention.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

### II. CLL Medicaments

In one aspect, the invention concerns selecting patients who can be treated with CLL medicaments based on expression of one or more of the biomarkers disclosed herein. Examples of CLL medicaments include, but are not limited to:

Chemotherapeutic agents and chemotherapy regimens noted above, including, in particular, alkylating agents (e.g. chlorambucil, bendamustine, or cyclophosphamide); nucleoside analogues or antimetabolites (e.g. fludarabine), fludarabine and cyclophosphamide (FC); prednisone or prednisolone; akylator-containing combination therapy, including cyclophosphamide, vincristine, prednisolone (CHOP), or cyclophosphamide, vincristine, prednisolone (CVP); etc.

B cell antagonists, such as CD20 antibodies (e.g. rituximab, ofatumumab, GA101, SBI-087, veltuzumab, and AME-133 etc), CD22 antibodies or CD79b antibodies;

Intravenous immune globulin;

CD52 antibodies (e.g. alemtuzumab); and

Other medicaments or combinations thereof in development, or approved, for treating CLL.

In one embodiment, the CLL medicament is one which induces FAK signaling and/or homotypic aggregation. The CLL medicament is optionally selected from: a B-cell antagonist, a B-cell antibody, a CD20 antibody, a Type I CD20 antibody, a Type II CD20 antibody, a CD22 antibody, a CD79b antibody, etc.

In one embodiment (where the biomarker expression level is below the median) the CLL medicament is one other than rituximab.

In one embodiment, the medicament is an antibody, e.g. directed against or which binds to CD20. The antibody herein includes: monoclonal antibodies, including a chimeric, humanized or human antibodies. In one embodiment, the antibody is an antibody fragment, e.g., a Fv, Fab, Fab′, scFv, diabody, or F(ab′)₂ fragment. In another embodiment, the antibody is a full length antibody, e.g., an intact IgG1 antibody or other antibody class or isotype as defined herein.

In one embodiment, the antibody, e.g. the antibody used in the methods herein may incorporate any of the features, singly or in combination, as described in Sections 1-6 below:

1. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

2. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5, 821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Rituximab is an example of a chimeric antibody that binds CD20. Examples of humanized antibodies that bind CD20 include: GA101, SBI-087, AME-133 and veltuzumab.

3. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Ofatumumab is an example of a human antibody that binds CD20.

4. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

5. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for CD20 and the other is for any other antigen. In certain embodiments, bispecific antibodies may bind to two different epitopes of CD20. Bispecific antibodies may also be used to localize cytotoxic agents to cells which express CD20. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see,e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to CD20 as well as another, different antigen (see, US 2008/0069820, for example).

6. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, antibody variants having one or more amino acid substitutions are provided. Sites of interest for substitutional mutagenesis include the HVRs and FRs. Amino acid substitutions may be introduced into an antibody of interest and the products screened for a desired activity, e.g., retained/improved antigen binding, decreased immunogenicity, or improved ADCC or CDC.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

GA101 is an example of an antibody glycosylation variant that binds CD20.

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious.

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

In one embodiment, the medicament is an immunoconjugate comprising an antibody (such as a CD20 antibody) conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### III. Diagnostic Methods

In one aspect, the invention herein concerns a method for selecting a therapy for a patient with chronic lymphocytic leukemia (CLL) comprising determining expression (or activation) of a biomarker selected from miRNA151 3p, miRNA409 3p, PTK2, and PI3K, in a sample from the patient, and selecting a CLL medicament based on the level of expression of the biomarker. In one embodiment, an elevated level of the biomarker(s) will result in selection of a CLL medicament which induces FAK signaling and/or homotypic adhesion, and/or a B-cell antagonist, and/or a CD20 antibody for use in treating the patient. In another embodiment, where the biomarker(s) are present a reduced level or a level below the median, the patient will be selected for a treatment with a CLL medicament other than rituximab or other than a CD20 antibody.

The median expression level for the biomarker can be determined essentially contemporaneously with measuring biomarker expression, or may have been determined previously. The skilled artisan is able to determine the median expression level of a biomarker in a population of patients for various diagnostic assays. This is exemplified in the table below which provides median expression levels for the various biomarkers and bioassays in the example below.

| | | | Number | | | | | |
|---|---|---|---|---|---|---|---|---|
| | | High vs low | of CD19 | | 1st | | 3rd | |
| Gene/miRNA | Data Type | expression definition | samples | Minimum | Quartile | Median | Quartile | Maximum |
| miRNA151 3p | DISCOVARRAY ®, RNA | high: >= median (6.77); | 301 | −0.01 | 3.33 | 6.77 | 8.96 | 11.67 |
| | normalized value (log2) | low: < median | | | | | | |
| | qRT-PCR, estimated Copy Number | high: >= median (58.6); | 281 | 0 | 6.59 | 58.6 | 237 | 3200 |
| | per ng | low: < median | | | | | | |
| | qRT-PCR, Ct | high: <= median (34.35); | 281 | 28.18 | 32.19 | 34.35 | 37.73 | 45 |
| | | low: > median | | | | | | |
| | qRT-PCR, -ΔCt relative to miR-150 | high: >= median (−13.85); | 280 | −25.77 | −17.23 | −13.85 | −12.06 | −8.87 |
| | | low: < median | | | | | | |
| | qRT-PCR, -ΔCt relative to miR-26a | high: >= median (−9.55); | 281 | −22.34 | −13.28 | −9.55 | −7.82 | −0.44 |
| | | low: < median | | | | | | |
| miRNA409 3p | DISCOVARRAY ®, RMA | high: >= median (1.12); | 301 | −0.27 | 0.58 | 1.12 | 2 | 7.46 |
| | normalized value (log2) | low: < median | | | | | | |
| | qRT-PCR, estimated Copy Number | high: >= median (0.38); | 281 | 0 | 0 | 0.38 | 1.74 | 181 |
| | per ng | low: < median | | | | | | |
| | qRT-PCR, Ct | high: <= median (39.03); | 281 | 30.35 | 36.9 | 39.03 | 45 | 45 |
| | | low: > median | | | | | | |
| | qRT-PCR, -ΔCt relative to miR-150 | high: >= median (−18.88); | 280 | −26.22 | −24.25 | −18.88 | −16.31 | −7.43 |
| | | low: < median | | | | | | |
| | qRT-PCR, -ΔCt relative to miR-26a | high: >= median (−14.84); | 281 | −22.34 | −20.17 | −14.84 | −12.33 | −0.44 |
| | | low: < median | | | | | | |
| PTK2 | Exon array (annotation version | high: >= median (5.15); | 300 | 3.79 | 4.13 | 5.15 | 6.21 | 8.16 |
| | na29.hg18), RMA normalized value | low: < median | | | | | | |
| | (log2) | | | | | | | |
| | qRT-PCR, Ct | high: <= median (30.65); | 272 | 25.92 | 28.43 | 30.65 | 34.71 | 40 |
| | | low: > median | | | | | | |
| | qRT-PCR, -ΔCt relative to 18S | high: >= median (−21.98); | 272 | −33.12 | −26.33 | −21.98 | −20.19 | −13.31 |
| | | low: < median | | | | | | |
| | qRT-PCR, -ΔCt relative to RPS6 | high: >= median (−12.58); | 272 | −20.45 | −12.58 | −8.22 | −6.42 | −1.81 |
| | | low: < median | | | | | | |
| | qRT-PCR, -ΔCt relative to HNRNPL | high: >= median (−9.56); | 272 | −17.39 | −9.56 | −5.24 | −3.7 | 1.5 |
| | | low: < median | | | | | | |
| | qRT-PCR, -ΔCt relative to UBC | high: >= median (−12.83); | 272 | −21.23 | −12.83 | −8.59 | −6.88 | −3.53 |
| | | low: < median | | | | | | |
| | qRT-PCR, -ΔCt relative to UBA52 | high: >= median (−12.26); | 272 | −19.84 | −12.26 | −8.12 | −6.29 | −2.76 |
| | | low: < median | | | | | | |

A sample from the patient is tested for expression of one or more of the biomarkers herein. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may contain compounds which are not naturally intermixed with the tissue in nature such as preservatives, anticoagulants, buffers, fixatives, nutrients, antibiotics, or the like. Examples of tumor samples herein include, but are not limited to, tumor biopsies, tumor cells, serum or plasma, Peripheral Blood Mononuclear Cells (PBMCs), circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples. In one embodiment the sample comprises Peripheral Blood Mononuclear Cells (PBMCs), including CD19-enriched PBMCs.

Various methods for determining expression of mRNA or protein include, but are not limited to, gene expression profiling, polymerase chain reaction (PCR) including quantitative real time PCR (qRT-PCR), RNA-Seq, microarray analysis, serial analysis of gene expression (SAGE), MassARRAY, proteomics, immunohistochemistry (IHC), etc. Preferably mRNA is quantified. Such mRNA analysis is preferably performed using the technique of polymerase chain reaction (PCR), or by microarray analysis. Where PCR is employed, a preferred form of PCR is quantitative real time PCR (qRT-PCR).

Various exemplary methods for determining gene expression will now be described in more detail.

1. Gene Expression Profiling

In general, methods of gene expression profiling can be divided into two large groups: methods based on hybridization analysis of polynucleotides, and methods based on sequencing of polynucleotides. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and polymerase chain reaction (PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

2. Polymerase Chain Reaction (PCR)

Of the techniques listed above, a sensitive and flexible quantitative method is PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE® Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP™ RNA PCR kit (Perkin Elmer, Calif., USA), following the manufacturer's instructions. The derived cDNA can then be used as a template in the subsequent PCR reaction. Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN® PCR can be performed using commercially available equipment, such as, for example, ABI PRISM 7700® Sequence Detection System® (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700® Sequence Detection System. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and P-actin.

A more recent variation of the PCR technique is quantitative real time PCR (qRT-PCR), which measures PCR product accumulation through a dual-labeled fluorigenic probe (i.e., TAQMAN® probe). Real time PCR is compatible both with quantitative competitive PCR, where internal competitor for each target sequence is used for normalization, and with quantitative comparative PCR using a normalization gene contained within the sample, or a housekeeping gene for PCR. For further details see, e.g. Held et al., Genome Research 6:986-994 (1996).

The steps of a representative protocol for profiling gene expression using fixed, paraffin-embedded tissues as the RNA source, including mRNA isolation, purification, primer extension and amplification are given in various published journal articles (for example: Godfrey et al., J. Molec. Diagnostics 2: 84-91 (2000); Specht et al., Am. J. Pathol. 158: 419-29 (2001)). Briefly, a representative process starts with cutting about 10 microgram thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR.

According to one aspect of the present invention, PCR primers and probes are designed based upon intron sequences present in the gene to be amplified. In this embodiment, the first step in the primer/probe design is the delineation of intron sequences within the genes. This can be done by publicly available software, such as the DNA BLAT software developed by Kent, W., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Rozen and Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

Factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g. about 50 to 70° C. are typically preferred.

For further guidelines for PCR primer and probe design see, e.g. Dieffenbach et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

3. RNN-Seq

RNA-Seq, also called Whole Transcriptome Shotgun Sequencing (WTSS) refers to the use of high-throughput sequencing technologies to sequence cDNA in order to get information about a sample's RNA content. Publications describing RNA-Seq include: Wang et al. “RNA-Seq: a revolutionary tool for transcriptomics”” Nature Reviews Genetics 10 (1): 57-63 (January 2009); Ryan et al. BioTechniques 45 (1): 81-94 (2008); and Maher et al. “Transcriptome sequencing to detect gene fusions in cancer”. Nature 458 (7234): 97-101 (January 2009).

4. Microarrays

Differential gene expression can also be identified, or confirmed using the microarray technique. Thus, the expression profile of breast cancer-associated genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the AFFYMETRIX GENCHIP™ technology, or Incyte's microarray technology.

The development of microarray methods for large-scale analysis of gene expression makes it possible to search systematically for molecular markers of cancer classification and outcome prediction in a variety of tumor types.

5. Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

6. MassARRAY Technology

The MassARRAY (Sequenom, San Diego, Calif.) technology is an automated, high-throughput method of gene expression analysis using mass spectrometry (MS) for detection. According to this method, following the isolation of RNA, reverse transcription and PCR amplification, the cDNAs are subjected to primer extension. The cDNA-derived primer extension products are purified, and dispensed on a chip array that is pre-loaded with the components needed for MALTI-TOF MS sample preparation. The various cDNAs present in the reaction are quantitated by analyzing the peak areas in the mass spectrum obtained.

7. Immunohistochemistry

Immunohistochemistry methods are also suitable for detecting the expression levels of the prognostic markers of the present invention. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

8. Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

Biomarker expression may also be evaluated using an in vivo diagnostic assay, e.g. by administering a molecule (such as an antibody) which binds the molecule to be detected and is tagged with a detectable label (e.g. a radioactive isotope) and externally scanning the patient for localization of the label.

### IV. Therapeutic Methods

In one embodiment, the invention provides a method for treating a patient with chronic lymphocytic leukemia (CLL), comprising administering a therapeutically effective amount of a CLL medicament to the patient if the patient has been found to have an elevated amount of one or more biomarker selected from miRNA151 3p, miRNA409 3p, and PTK2; or reduced PI3K biomarker. Examples of CLL medicaments herein include: a CLL medicament which induces FAK signaling and/or induce homotypic adhesion; B-cell antagonists or B-cell antibodies; CD20 antibodies (including humanized, human, chimeric, Type I or Type II anti-CD20 antibodies, such as rituximab, ofatumumab, GA101, SBI-087, veltuzumab, and AME-133).

The invention also concerns a method for treating a patient with chronic lymphocytic leukemia (CLL) comprising administering to the patient a therapeutically effective amount of an anti-CD20 antibody (e.g. rituximab), if the patient has been found to have an elevated amount of one or more biomarker selected from miRNA151 3p, miRNA409 3p, and PTK2; or reduced PI3K biomarker.

Moreover, the invention provides a method for treating a patient with chronic lymphocytic leukemia (CLL) comprising administering to the patient a therapeutically effective amount of a combination of rituximab, fludarabine and cyclophosphamide, if the patient has been found to have an elevated amount of one or more biomarker selected from miRNA151 3p, miRNA409 3p, and PTK2; or reduced PI3K biomarker.

The invention additionally concerns a method for treating a patient with chronic lymphocytic leukemia (CLL) comprising administering to the patient a therapeutically effective amount of CLL medicament other than rituximab, if the patient has been found to have a reduced amount of one or more biomarker selected from miRNA151 3p, miRNA409 3p, and PTK2; or elevated PI3K biomarker.

The patient treated herein desirably will benefit from greater progression free survival (PFS) relative to a patient who does not have an elevated amount of the biomarker.

CLL medicaments of the invention can be used either alone or in combination with other CLL medicaments. For instance, a CD20 antibody may be co-administered with at least one additional therapeutic agent, e.g. with a chemotherapy regimen including, in particular, alkylating agents (e.g. chlorambucil, bendamustine, or cyclophosphamide), nucleoside analogues or antimetabolites (e.g. fludarabine), fludarabine and cyclophosphamide (FC), prednisone or prednisolone, akylator-containing combination therapy, including cyclophosphamide, vincristine, prednisolone (CHOP), or cyclophosphamide, vincristine, prednisolone (CVP), etc, with other B cell antagonists, such as CD20 antibodies (e.g. rituximab, ofatumumab, GA101, SBI-087, veltuzumab, and AME-133 etc), CD22 antibodies or CD79b antibodies, with intravenous immune globulin; and/or with CD52 antibodies (e.g. alemtuzumab). Such combination therapies noted above encompass combined administration (where two or more therapeutic agents are included in the same or separate formulations), and separate administration, in which case, administration of a first medicament can occur prior to, simultaneously, and/or following, administration of a second medicament. CLL medicaments of the invention can also be used in combination with radiation therapy.

The medicament(s) herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. An exemplary dosage regimen for a CD20 antibody includes weekly, biweekly, or monthly administrations of the antibody in the range from about 500 mg/m² to about 1500 mg/m². An exemplary dosage regimen for rituximab is 375 mg/m² (day 1) then 500 mg/m² (cycles 2-6) once every 28 days. Exemplary dosage regiments for ofatumumab: 300 mg initial dose followed by 2000 mg dose (every month); repeated doses of 500 mg or 1000 mg; 300 mg with 1000 mg 1 week later, followed by up to 11 monthly infusions of 1000 mg, etc.

However, other dosage regimens may be useful. The progress of this therapy is easily monitored by conventional techniques and assays.

It is understood that any of the above formulations or therapeutic methods may be carried out using an immunoconjugate of the invention in place of or in addition to an antibody as the medicament.

### V. Articles of Manufacture

In another embodiment of the invention, an article of manufacture for use in treating CLL. The article of manufacture comprises a container and a label or package insert on or associated with the container. Suitable containers include, for example, bottles, vials, syringes, etc. The containers may be formed from a variety of materials such as glass or plastic. The container holds or contains a composition comprising the CLL medicament as the active agent and may have a sterile access port (for example the container may be an intravenous solution bag or a vial having a stopper pierceable by a hypodermic injection needle).

The article of manufacture may further comprise a second container comprising a pharmaceutically-acceptable diluent buffer, such as bacteriostatic water for injection (BWFI), phosphate-buffered saline, Ringer's solution and dextrose solution. The article of manufacture may further include other materials desirable from a commercial and user standpoint, including other buffers, diluents, filters, needles, and syringes.

The article of manufacture of the present invention also includes information, for example in the form of a package insert, indicating that the composition is used for treating CLL based on expression level of the biomarker(s) herein. The insert or label may take any form, such as paper or on electronic media such as a magnetically recorded medium (e.g., floppy disk) or a CD-ROM. The label or insert may also include other information concerning the pharmaceutical compositions and dosage forms in the kit or article of manufacture.

According to one embodiment of the invention, an article of manufacture is provided comprising, packaged together, a CLL medicament (e.g. B-cell antagonist, or anti-CD20 antibody) in a pharmaceutically acceptable carrier and a package insert indicating that the CLL medicament is for treating a patient with chronic lymphocytic leukemia (CLL) based on expression of one or more biomarker selected from miRNA151 3p, miRNA409 3p, PTK2, and PI3K.

The invention also concerns a method for manufacturing an article of manufacture comprising combining in a package a pharmaceutical composition comprising a CLL medicament (e.g. B-cell antagonist, or anti-CD20 antibody) and a package insert indicating that the pharmaceutical composition is for treating a patient with chronic lymphocytic leukemia (CLL) based on expression of one or more biomarker selected from miRNA151 3p, miRNA409 3p, PTK2, and PI3K.

The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

### VI. Diagnostic Kits

The invention also concerns diagnostic kits useful for detecting any one or more of the biomarker(s) identified herein. Accordingly, a diagnostic kit is provided which comprises one or more reagents for determining expression of a biomarker selected from miRNA151 3p, miRNA409 3p, PTK2, and PI3K in a sample from a CLL patient. Optionally, the kit further comprises instructions to use the kit to select a CLL medicament (e.g. B-cell antagonist, or anti-CD20 antibody) for treating the CLL patient if the patient expresses the biomarker at an elevated level. In another embodiment, the instructions are to use the kit to select a CLL medicament other than rituximab (or other than an anti-CD20 antibody) if the patient expresses the biomarker at a reduced level. In one embodiment, e.g. of a PCR kit, the one or more reagents comprise a pair of DNA primers and probe for detecting the miRNA151 3p, miRNA409 3p, PTK2, or PI3K biomarker.

### VII. Methods of Advertising

The invention herein also concerns a method for advertising a CLL medicament comprising promoting, to a target audience, the use of the CLL medicament (e.g. B-cell antagonist, or anti-CD20 antibody) for treating a patient with chronic lymphocytic leukemia (CLL) based on expression of one or more biomarker selected from miRNA151 3p, miRNA409 3p, PTK2, and PI3K.

Advertising is generally paid communication through a non-personal medium in which the sponsor is identified and the message is controlled. Advertising for purposes herein includes publicity, public relations, product placement, sponsorship, underwriting, and sales promotion. This term also includes sponsored informational public notices appearing in any of the print communications media designed to appeal to a mass audience to persuade, inform, promote, motivate, or otherwise modify behavior toward a favorable pattern of purchasing, supporting, or approving the invention herein.

The advertising and promotion of the diagnostic method herein may be accomplished by any means. Examples of advertising media used to deliver these messages include television, radio, movies, magazines, newspapers, the internet, and billboards, including commercials, which are messages appearing in the broadcast media. Advertisements also include those on the seats of grocery carts, on the walls of an airport walkway, and on the sides of buses, or heard in telephone hold messages or in-store PA systems, or anywhere a visual or audible communication can be placed.

More specific examples of promotion or advertising means include television, radio, movies, the internet such as webcasts and webinars, interactive computer networks intended to reach simultaneous users, fixed or electronic billboards and other public signs, posters, traditional or electronic literature such as magazines and newspapers, other media outlets, presentations or individual contacts by, e.g., e-mail, phone, instant message, postal, courier, mass, or carrier mail, in-person visits, etc.

The type of advertising used will depend on many factors, for example, on the nature of the target audience to be reached, e.g., hospitals, insurance companies, clinics, doctors, nurses, and patients, as well as cost considerations and the relevant jurisdictional laws and regulations governing advertising of medicaments and diagnostics. The advertising may be individualized or customized based on user characterizations defined by service interaction and/or other data such as user demographics and geographical location.

### VIII. Deposit of Biological Material

The following biological material has been deposited with the American Type Culture Collection, 10801 University Boulevard, Manassas, Va. 20110-2209, USA (ATCC):

| Deposit | ATCC No. | Deposit Date |
|---|---|---|
| DNA encoding rituximab | 69119 | 10 Nov. 1992 |
| (in TCAE vector) | | |

### EXAMPLES

### Biomarkers Predicting Response of CLL to CD20 Antibodies

### Materials and Methods

### Samples:

Pretreatment patient samples were analyzed from an international, multicenter, open-label, phase III trial, randomizing CLL patients to receive R-FC (rituximab plus fludarabine/cyclophosphamide) or FC (fludarabine/cyclophosphamide) alone. The primary objective was to demonstrate superior progression free survival (PFS) for R-FC compared with FC alone. The study protocol was approved by institutional review boards at participating centers and all patients gave written informed consent. Details on trial design and eligibility criteria have been described in Robak et al. J. Clin. Oncol. 28(10):1756-1765 (2010). Patients were selected on the availability of RNA and the written informed consent to participate in molecular genetic analyses of peripheral blood samples.

Pretreatment Peripheral Blood Mononuclear Cell (PBMC) samples positively selected for CD19+ cells were available from n=301 patients. CD19 separation was performed by magnetic bead separation according to the manufacturer's protocol (Miltenyi, Germany).

### RNA Isolation:

Human cell pellets were homogenized in Qiagen Buffer RLT. Total RNA was isolated from human cell pellet homogenates by Asuragen, Inc., according to the company's standard operating procedure, optimized for retention of microRNAs and including a DNAse treatment step. The purity and quantity of total RNA samples were determined by absorbance readings at 260 and 280 nm using a NanoDrop ND-1000 UV spectrophotometer. The integrity of total RNA was qualified by Agilent Bioanalyzer 2100 capillary electrophoresis, using the Nano Assay. Sample suitability for microRNA profiling was determined by singleplex qRT-PCR for 1 to 2 proprietary microRNAs as a surrogate measure of overall microRNA abundance

miRNA Expression Profiling:
DISCOVARRAY® miRNA Profiling Service Platform.

A custom-manufactured AFFYMETRIX GENECHIP® from Ambion was designed to miRNA probes derived from the Sanger miRBase (Marcus et al., J Clin Oncol. 26:4579-4586 (2008); Hiddemann et al., Blood 106:3725-3732 (2005); http://microrna.sanger.ac.uk/sequences/index.shtml) and published reports (Coiffier et al., N. Engl J. Med. 346:235-242 (2002); Feugier et al., J Clin Oncol. 23(18):4117-4126 (2005); and Hallek et al., Blood, ASH Annual Meeting Abstract, 112: 325 (2008); and Robak et al., Blood, ASH Annual Meeting Abstracts, 112:lba-1 (2008)). Antigenomic probe sequences were provided by AFFYMETRIX® and derived from a larger set of controls used on the AFFYMETRIX® human exon array for estimating background signal, as described below. Other non-miRNA control probes on the array were designed to lack sequence homology to the human genome and can be used for spike-in external reference controls.

### Sample and Array Processing:

Samples for miRNA profiling studies were processed by Asuragen Services (Austin, Tex.), according to the company's standard operating procedures. Following sample quality control (QC) assessment, the 3′ ends of RNA molecules in total RNA samples (400 ng total RNA per sample) were labeled with biotin according to the company's standard protocol. Hybridization, washing, staining, imaging, and signal extraction were performed according to AFFYMETRIX®-recommended procedures, except that the 20× GENECHIP® Eukaryotic Hybridization Controls were omitted from the hybridization.

### Signal Processing:

The signal processing implemented for the Ambion miRChip is a multi-step process involving probe specific signal detection calls, background estimate and correction, constant variance stabilization and either array scaling or global normalization (Hallek et al., Blood, ASH Annual Meeting Abstract, 112: 325 (2008)). For each probe, an estimated background value is subtracted that is derived from the median signal of a set of G-C-matched anti-genomic controls. Arrays within a specific analysis experiment are normalized together according to the variance stabilization method described by Hallek et al., Blood, ASH Annual Meeting Abstracts, 114: Abstract 535 (2009). Detection calls are based on a Wilcoxon rank-sum test of the miRNA probe signal compared to the distribution of signals from GC-content matched anti-genomic probes.

### Statistical Analysis:

For statistical hypothesis testing, a two-sample t-test, with assumption of equal variance, was applied. One-way ANOVA was used for experimental designs with more than two experimental groupings or levels of the same factor. These tests define which probes are considered to be differentially expressed based on two criteria: a default p-value of 0.001 and log₂ difference>1.

### AFFYMETRIX® U133 Plus 2.0:

Gene expression profiling was performed using AFFYMETRIX® Human U133 Plus 2.0 full genome oligonucleotide arrays. RNA sample labeling was performed using the AFFYMETRIX® GENECHIP® Array Station (GCAS) according manufacture automated protocol. In brief, biotinylated cRNA was generated starting with 0.5 μg of total RNA from each sample. 2 μl of 22.5 μM mixture of 5 Gene Logic's globin reduction oligos comprised of 2-alpha, 2-beta, and 1-gamma hemoglobin gene were added to the total RNA reaction. The globin oligomers are gene-specific blockers that greatly reduce the amount of globin cDNAs generated from globin mRNA during the first-strand cDNA synthesis. Roche purchased the oligomer sequences from GeneLogic (Gainthersburg Md., USA).

On the GENECHIP® Array System (GCAS) robot, the target labeling method was carried out with the GENECHIP® HT One-Cycle Target Labeling kit P/N 900686. Samples were then stained, washed and hybridized to AFFYMETRIX® arrays according to manufacture protocol. Samples were hybridized in batches of 24 samples. Arrays were scanned using GENECHIP® Scanner 3000. AFFYMETRIX® GENECHIP® Operation Software (GCOS) was used to capture raw signal intensities. Microarray Suite 5.0 (MAS5.0) and Expression Console were used for basic computational analysis.

Further statistical analyses were performed suing RMA algorithm which summarized the probe intensities into probeset signal (Irizarry et al., Biostatistics 4(2): 249-264 (2003)). Probe set signal intensities were normalized using a quantile-quantile method (Bolstad et al., Bioinformatics 19:185-193 (2003)). All normalized data were log 2-transformed prior to analysis to down-weight the influence of high expression values.

After applying standard procedures of quality control (Irizarry et al. supra), 240 samples were finally selected for statistical analysis.

Of the potential 54,675 probe sets present on the U133 Plus 2.0 microarray, 41,256 probe sets were present in at least two samples where the presence call was defined by the MAS5.0 algorithm (AFFYMETRIX® Statistical Algorithms Description Document, 2002). Statistical analysis was performed on the set of present probe sets.

AFFYMETRIX® gene expression profiling of Total RNA using Whole-Transcript Assay (WTA) and Gene/Exon 1.0 ST Arrays.

Samples for mRNA profiling studies were processed by Asuragen, Inc. according to the company's standard operating procedures. Biotin-labeled sense strand cDNA was prepared from 1 μg total RNA per sample using a modified AFFYMETRIX GENECHIP® Whole Transcript (WT) Sense Target Labeling Assay (AFFYMETRIX®, Inc.). Intermediate cRNA and resulting cDNA yields were quantified by spectrophotometry. Fragmentation and labeling of cDNA was performed using 5 μg for Exon Arrays. Hybridization to arrays was carried out at 45° C. for 16 hours in an AFFYMETRIX® Model 640 hybridization oven. Arrays were washed and stained on an AFFYMETRIX® FS450 Fluidics station. The arrays were scanned on an AFFYMETRIX® GENECHIP® Scanner 3000 7G. For every array scanned, .DAT, .CEL, .jpg, and .xml flat files are provided. In addition, RMA normalized data is provided for the core dataset and the corresponding QC information which captures metrics including Area Under the Curve (AUC) and polyA spikes.

qRT-PCR Reverse Transcription, Amplification, and Analysis, Reverse Transcription and PCR Amplification:

Stock ABI Taqman miRNA assays, PCR master mixes, and reverse transcription (RT) components were used. 1 ng total RNA, in a volume of 4 μL, is reverse transcribed in a total reaction volume of 10 μL for each replicate of each assay. 3 μL of the RT product is carried forward into each 15 μL Taqman PCR amplification. All amplifications are performed on a validated ABI 7900HT real-time thermocycler. Known copy number synthetic miRNAs are included as positive controls for each assay set. Average Ct values from each set of replicates are compared to independently-generated standard curves derived from known copy number synthetic RNAs diluted in yeast tRNA. Standard curves are generated for synthetic RNA templates between 500 and 50,000,000 copies/reaction. R̂2 values are reported for each standard curve.

Results for PTK2 were confirmed by qRT-PCR technology using ABI kits according to the manufacturer's protocol using UBC as control gene.

Results for miRNA151 3p and miRNA 409 3p were confirmed by qRT-PCR technology using ABI kits according to the manufacturer's protocol. Expression levels of miRNA151 3p and miRNA409 3p were analyzed relative to the control genes miRNA 150 and miRNA 26 a that showed universally high expression levels.

### Statistical Analysis

Clinical Data

Pretreatment clinical features i.e demographics, prognostic markers (cytogenetic aberrations like del(17p), del(11q), ZAP70 and CD38 expression, IgVH status), progression free survival (PFS) were compared using the Fisher's exact, Mann-Whitney, or log-rank tests. A p-value of <0.05 was considered statistically significant.

Cox Regression Modeling and Testing

Two approaches using a Cox regression model were used to identify genes and miRNA associated with survival. First approach considered the models below.

### Model A

hᵢ(t)=exp(β₁Tx+β₂RNA)h₀(t)

### Model B

hᵢ(t)=exp(β₁Tx+β₂Age+β₃Binet+β₄IgVH+β₅Del17p+β₆Del11q+β₇RNA)h₀(t)

where
hᵢ(t) is the hazard function of the individual i
hₒ(t) is the baseline hazard function
βᵢ's are the coefficients of the following explanatory variables:
   Age, Binet, IgVH, Del17p, del11q and Tx which respectively code for age, Binet Stage, IgVH, Del17p and del11q mutational status and treatment group
   RNA which represents the probe set signal intensity (log-2 transformed)

For both models, the null hypothesis was there was no relationship between the probe set intensity signal and survival.

The second approach used the two models below (C and D), comparing them with a log-Likelihood ratio test (LRT test). Being interested in the possible ability of the probe set signal at baseline to predict for a better survival in one treatment group compared to the other, we included both terms in the model B: probe set signal intensity and the interaction term of the probe set signal intensity with the treatment factor.

Model C

hᵢ(t)=exp(β₁Tx+β₂Age+β₃BinetStage+β₄IgVH+β₅Del17p)h₀(t)

Model D

hᵢ(t)=exp(β₁Tx+β₂Age+β₃BinetStage+β₄IgVH+β₅Del17p+β₆RNA+β₇Tx:RNA)h₀(t)

The null hypothesis was there was no relationship between probe set intensity and survival, the alternative hypothesis being there was such a relationship and moreover differing between both treatment groups.

The two tests (Wald and LRT) were run for each probe set identified as present. Due to the large number of tests performed, a false discovery rate (FDR) based multiple testing procedure (Storey et al., Proceedings of the National Academy of Sciences 100:9440-9445 (2003)) was applied to control the expected proportion of genes (or miRNA) erroneously identified as differentially expressed by both Wald and log-Likelihood ratio tests. A FDR of 10% was chosen to declare a significant difference.

For the different tables and graphs appearing in this document where median PFS was computed, mRNA and miRNA signal intensities were dichotomized using the median value as a cutoff.

miRNA Anti-Correlations and 3′UTR Luciferase Assays

Predicted targets were downloaded from TARGETSCAN 5.1™ (http://www.targetscan.org/) and expression was extracted from the mRNA expression profiling data, irrespective of predicted site conservation. Anti-correlations with miRNAs were determined by comparing miRNA array and mRNA array data and computing Pearson correlation coefficients. Significantly anti-correlated targets were determined with a q-value<0.01. Subsequently, 3′UTRs that met this threshold were cloned upstream into 3′UTR luciferase reporter constructs (Switchgear Genomics) and 100 ng of each construct was transfected into HeLa cells (ATCC) with 25 ng pRL-CMV, 10 nM miRNA151 3p mimic (Dharmacon) or scrambled mimic in 96-well plates. Luciferase activity was determined 24 hours post-transfection and all data were normalized to renilla luciferase transfection control and scrambled control mimic. Three biological replicates were performed. Statistical significance for a difference between scrambled control and miRNA151 3p mimic was determined by a 2-sided Student's t-test.

### Results

### Micro RNA Data:

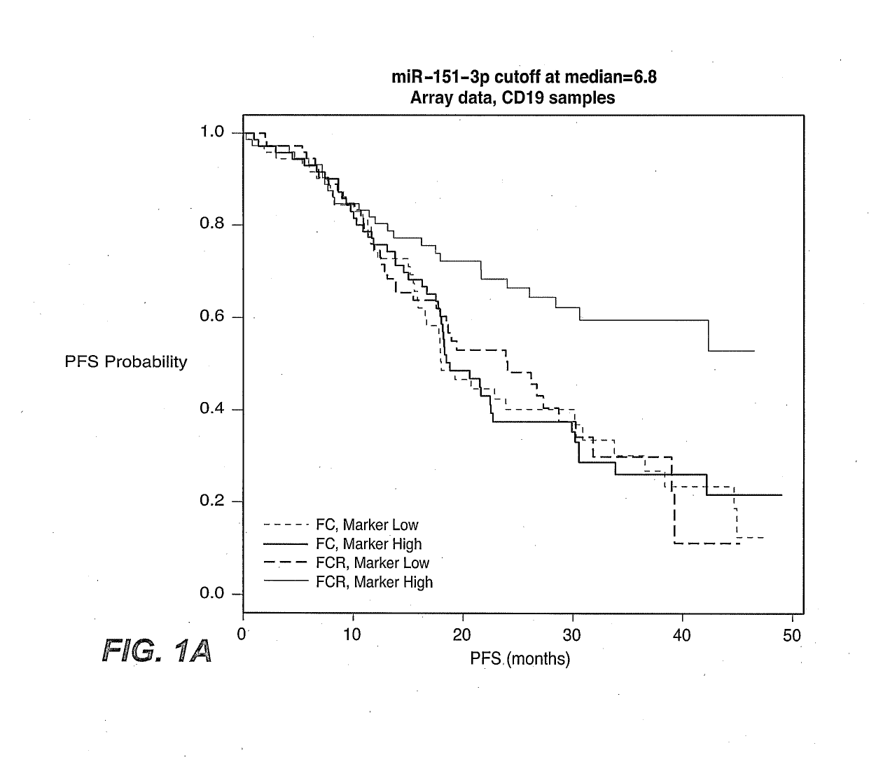

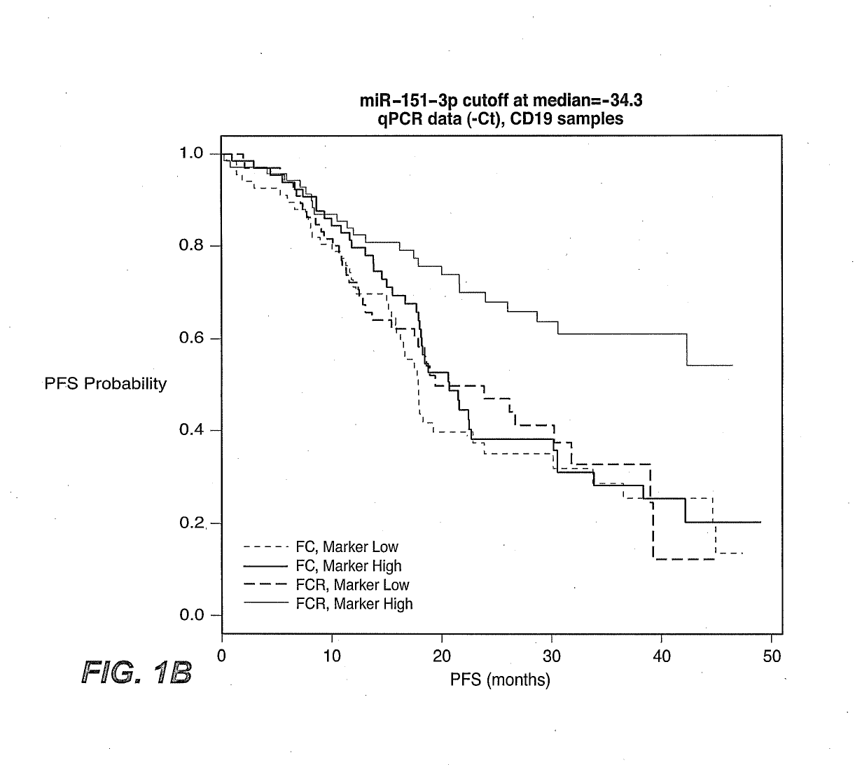

Using the DISCOVARRAY® microRNA platform miRNA151 3p and miRNA 409-3p emerged as significant predictive miRNAs for PFS in Rituximab based therapy (FIGS. 1A, 1B and 2A, 2B, Table 1, 2A, 2B).

Validation of array data by qRT-PCR confirmed the predictive significance of miRNA 151 3p and miRNA 409 3p (FIGS. 2A and 2B, Tables 1, 2A, 2B).

The independent predictive significance remained when incorporating both in a joint multivariate model including pretreatment factors that demonstrated prognostic significance in the study cohort (treatment FC vs. FCR, Age, Binet stage, IgVH mutational status, del(17p)).

Treatment: miRNA151 3p interaction: HR 1.09 (1.01-1.17), p=0.021.

Treatment: miRNA409 3p interaction: HR 1.09 (1.1-1.18), p=0.047.

Combination of miRNA151 3p and miRNA409 3p in the same model did not reveal additive predictive significance.

TABLE 1
Median PFS with respect to treatment and miRNA151 3p, miRNA409 3p expression

| | Median PFS | Median PFS | Median PFS | Median PFS |
|---|---|---|---|---|
| | miRNA151 | miRNA151 | miRNA409 | miRNA409 |
| | DISCOVARRAY ® | qRT-PCR | DISCOVARRAY ® | qRT-PCR |
| Tx: FC, | 18 | 17.9 | 21.6 | 18.3 |
| miRNA signal < | | | | |
| median miRNA signal | | | | |
| Tx: FC, | 18.5 | 20.7 | 18 | 18.3 |
| miRNA signal > | | | | |
| median miRNA signal | | | | |
| Tx: FCR, | 24 | 23.9 | 24 | 26.2 |
| miRNA signal < | | | | |
| median miRNA signal | | | | |
| Tx: FCR, | Not reached | Not reached | 39.3 | Not reached |
| miRNA signal > | | | | |
| median miRNA signal | | | | |

TABLE 2A
Predictive significance of miRNA151 3p for
PFS in anti-CD20 based therapy in CLL

| | Array | | RT-PCR | |
|---|---|---|---|---|
| | HR | P | HR | P |
| Tx: FC, miRNA151 < | 0.99 | 0.96 | 0.84 | 0.43 |
| median vs > median | | | | |
| Tx: FCR, miRNA151 > | 0.49 | 0.005 | 0.47 | 0.0049 |
| median vs > median | | | | |
| miRNA151 > median: | 0.47 | 0.0019 | 0.46 | 0.0027 |
| Tx FCR vs FC | | | | |
| miRNA151 < median: | 0.98 | 0.92 | 0.87 | 0.55 |
| Tx: FCR vs FC | | | | |

TABLE 2B
Predictive significance of miRNA409 3p for
PFS in anti-CD20 based therapy in CLL

| | Array | | RT-PCR | |
|---|---|---|---|---|
| | HR | P | HR | P |
| Tx: FC, miRNA409 < | 1.12 | 0.6 | 1.03 | 0.88 |
| median vs > median | | | | |
| Tx: FCR, miRNA409 > | 0.5 | 0.0052 | 0.54 | 0.025 |
| median vs > median | | | | |
| miRNA409 > median: | 0.44 | 0.00098 | 0.44 | 0.0028 |
| Tx FCR vs FC | | | | |
| miRNA409 < median: | 1 | 0.98 | 0.81 | 0.35 |
| Tx: FCR vs FC | | | | |

As shown in FIG. 1A, patients with higher than median expression level of miRNA151 3p had significantly longer PFS when treated with FCR (n=75, events: 26, median PFS not reached) as compared to FC (n=76, events: 47, median PFS: 18.5 months; p=0,0019). Patients treated with FCR had significantly longer PFS when initial miRNA151 3p level was higher than median (n=75, events: 26, median PFS not reached) as compared to lower than median expression levels of miRNA151 3p (n=74, events: 41, median PFS 24 months; p=0.005). No differences in PFS were observed in patients treated with FC regardless of miRNA151 3p expression levels (p=0.96). No differences in PFS were observed in lower than median miRNA151 3p expressers regardless of therapy (p=0.92)

As shown in FIG. 1B, patients with higher than median expression level of miRNA151 3p had significantly longer PFS when treated with FCR (n=71, events: 25, median PFS not reached) as compared to FC (n=69, events: 41, median PFS: 20.7 months; p=0.0027). Patients treated with FCR had significantly longer PFS when initial miRNA151 3p level was higher than median (n=71, events: 25, median PFS not reached) as compared to lower than median expression levels of miRNA151 3p (n=66, events: 36, median PFS 23.9 months; p=0.0049). No differences in PFS were observed in patients treated with FC regardless of miRNA151 3p expression levels (p=0.43). No differences in PFS were observed in lower than median miRNA151 3p expressers regardless of therapy (p=0.55)

FIG. 2B shows that patients with higher than median expression level of miRNA409 3p had significantly longer PFS when treated with FCR (n=75, events: 27, median PFS not reached) as compared to FC (n=76, events: 43, median PFS: 18 months; p=0.00098). Patients treated with FCR had significantly longer PFS when initial miRNA409 3p level was higher than median (n=75, events: 27, median PFS not reached) as compared to lower than median expression levels of miRNA409 3p (n=74, events: 40, median PFS 24 months; p=0.0052). No differences in PFS were observed in patients treated with FC regardless of miRNA409 3p expression levels (p=0.6). No differences in PFS were observed in lower than median miRNA409 3p expressers regardless of therapy (p=0.98).

FIG. 2B shows that patients with higher than median expression level of miRNA409 3p had significantly longer PFS when treated with FCR (n=64, events: 20, median PFS not reached) as compared to FC (n=76, events: 43, median PFS: 18.3 months; p=0.0028). Patients treated with FCR had significantly longer PFS when initial miRNA409 3p level was higher than median (n=64, events: 20, median PFS not reached) as compared to lower than median expression levels of miRNA409 3p (n=73, events: 41, median PFS 26.2 months; p=0.025). No differences in PFS were observed in patients treated with FC regardless of miRNA409 3p expression levels (p=0.88). No differences in PFS were observed in lower than median miRNA409 3p expressers regardless of therapy (p=0.35)

mRNA Data:

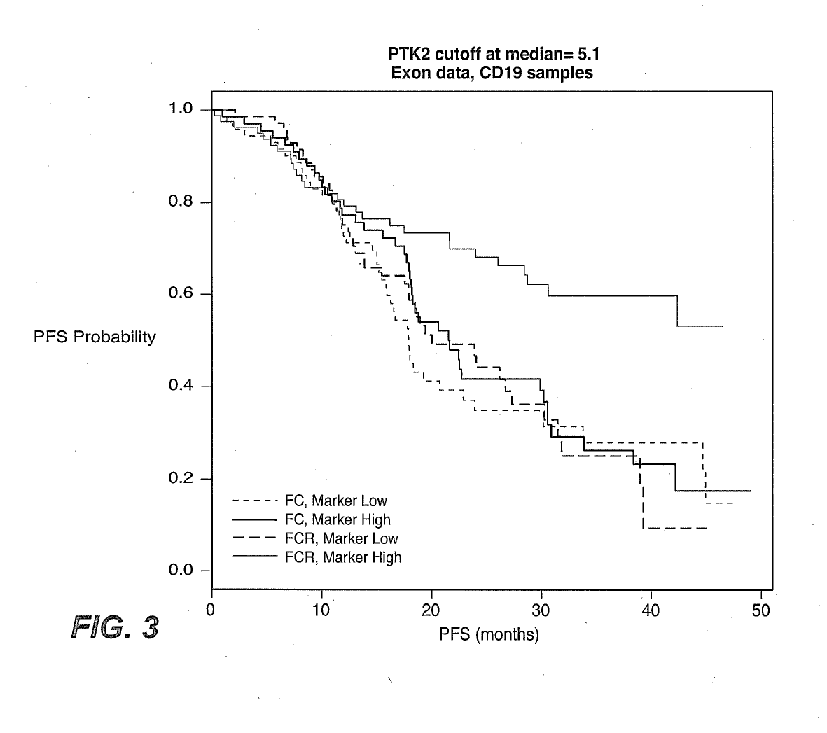

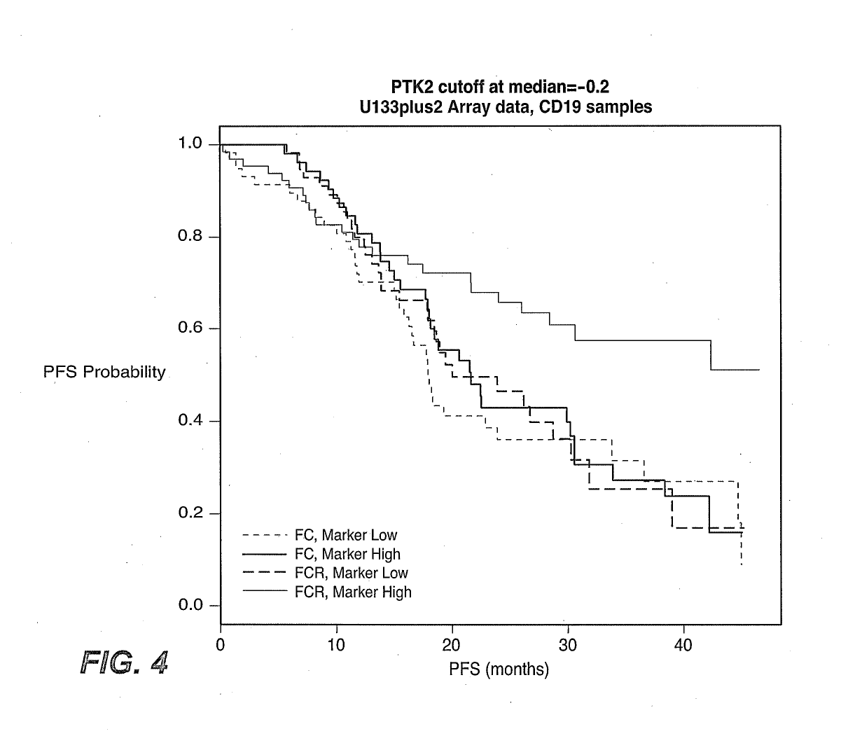

Using the AFFYMETRIX Exon 1.0 ST and U133 plus 2.0® microarray platforms PTK2 emerged as significant predictive differentially expressed gene for PFS in rituximab based therapy (see FIGS. 3 and 4; Tables 3 and 4). PTK2 is associated with progression free survival in multivariate analysis including prognostic factors (treatment FC vs FCR, Age, Binet stage, IgVH mutational status, del(17p)). WALD test remained significant after correction for multiple testing (P-value=1.5 10⁻⁴−q-value<0.1)

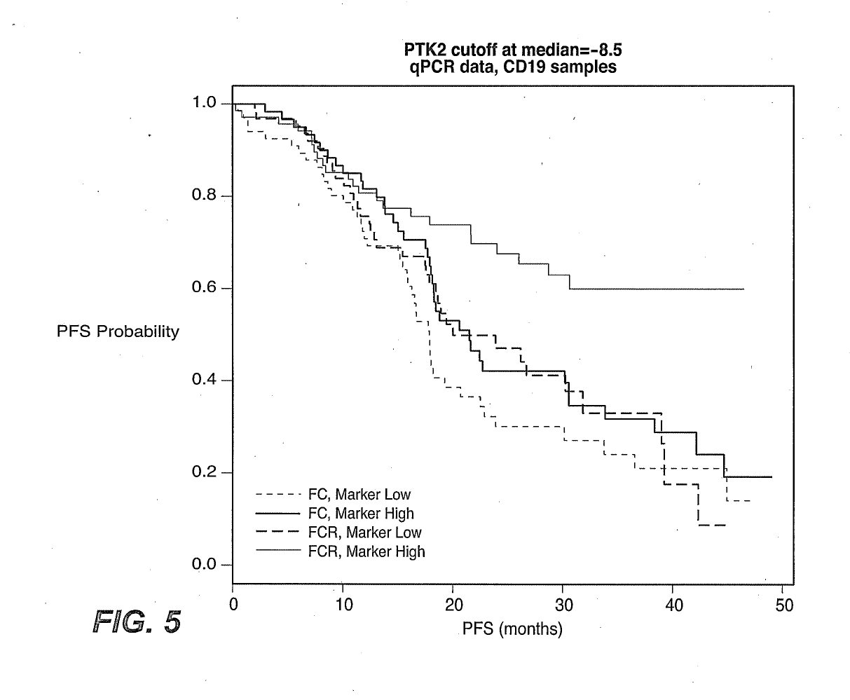

Validation of array data by qRT-PCR confirmed the predictive significance of PTK2 (FIG. 5; Tables 3 and 4).

Furthermore miRNA 151 is intronic to PTK2 on the chromosome 8 and most-probably co-expressed, correlation between their expression levels being very high (Pearson Correlation coefficient=0.84).

TABLE 3
Median PFS (months) with respect to treatment
and PTK2 expression in CD19+ cells according
to AFFYMETRIX ® array platforms.

| | Median PFS | Median PFS | Median PFS |
|---|---|---|---|
| | PTK2 | PTK2 | PTK2 |
| | Exon 1.0 ST | U133 plus2.0 | qRT-PCR |
| Tx: FC, gene < median | 17.9 | 17.9 | 17.9 |
| Tx: FC, gene > median | 21.5 | 21.5 | 21.5 |
| Tx: FCR, gene < median | 20 | 20 | 20 |
| Tx: FCR, gene > median | Not reached | 42.4 | Not reached |

TABLE 4
Predictive significance of PTK2 expression in CD19+ cells with
respect to treatment (FC vs FCR) according to
AFFYMETRIX ® array platforms.

| | Exon | | | | qRT- | |
|---|---|---|---|---|---|---|
| | 1.0 ST | | U133 + 2 | | PCR | |
| | HR | P | HR | P | HR | P |
| Tx: FC, PTK2 < | 0.86 | 0.48 | 0.86 | 0.53 | 0.77 | 0.25 |
| median vs > | | | | | | |
| median | | | | | | |
| Tx: FCR, PTK2 > | 0.46 | 0.0016 | 0.55 | 0.027 | 0.47 | 0.0049 |
| median vs > | | | | | | |
| median | | | | | | |
| PTK2 > median: | 0.48 | 0.0031 | 0.51 | 0.011 | 0.48 | 0.0059 |
| Tx FCR vs FC | | | | | | |
| PTK2 < median: | 0.94 | 0.79 | 0.83 | 0.46 | 0.82 | 0.37 |
| Tx: FCR vs FC | | | | | | |

FIG. 3 shows that patients with higher than median expression level of PTK2 had significantly longer PFS when treated with FCR (n=81, events: 28, median PFS not reached) as compared to FC (n=69, events: 42, median PFS: 21.5 months; p=0.0031). Patients treated with FCR had significantly longer PFS when initial PTK2 expression level was higher than median (n=81, events: 28, median PFS not reached) as compared to lower than median expression levels of PTK2 (n=71, events: 42, median PFS 20 months; p=0.0016). No differences in PFS were observed in patients treated with FC regardless of PTK2 expression levels (p=0.48). No differences in PFS were observed in lower than median PTK2 expressers regardless of therapy (p=0.79)

FIG. 4 shows that patients with higher than median expression level of PTK2 had significantly longer PFS when treated with FCR (n=67, events: 25, median PFS 42.4 months) as compared to FC (n=52, events: 34, median PFS: 21.5 months; p=0.011). Patients treated with FCR had significantly longer PFS when initial PTK2 expression level was higher than median (n=67, events: 25, median PFS 42.4 months) as compared to lower than median expression levels of PTK2 (n=55, events: 31, median PFS 20 months; p=0.027). No differences in PFS were observed in patients treated with FC regardless of PTK2 expression levels (p=0.53). No differences in PFS were observed in lower than median PTK2 expressers regardless of therapy (p=0.46)

FIG. 5 shows that patients with higher than median expression level of PTK2 had significantly longer PFS when treated with FCR (n=71, events: 23, median PFS not reached) as compared to FC (n=65, events: 39, median PFS: 21.5 months; p=0.0059). Patients treated with FCR had significantly longer PFS when initial PTK2 expression level was higher than median (n=71, events: 23, median PFS not reached) as compared to lower than median expression levels of PTK2 (n=63, events: 36, median PFS 20 months; p=0.0049). No differences in PFS were observed in patients treated with FC regardless of PTK2 expression levels (p=0.25). No differences in PFS were observed in lower than median PTK2 expressors regardless of therapy (p=0.37).

To assess the potential biological consequence of altered expression levels of miRNA151 3p the expression of these miRNAs was correlated with the mRNA expression of their predicted target genes, conserved and non-conserved within the 3′UTR, since miRNAs are primarily thought to exert their effects at the level of mRNA degradation. Guo et al. Nature 466(7308):835-40 (2010).

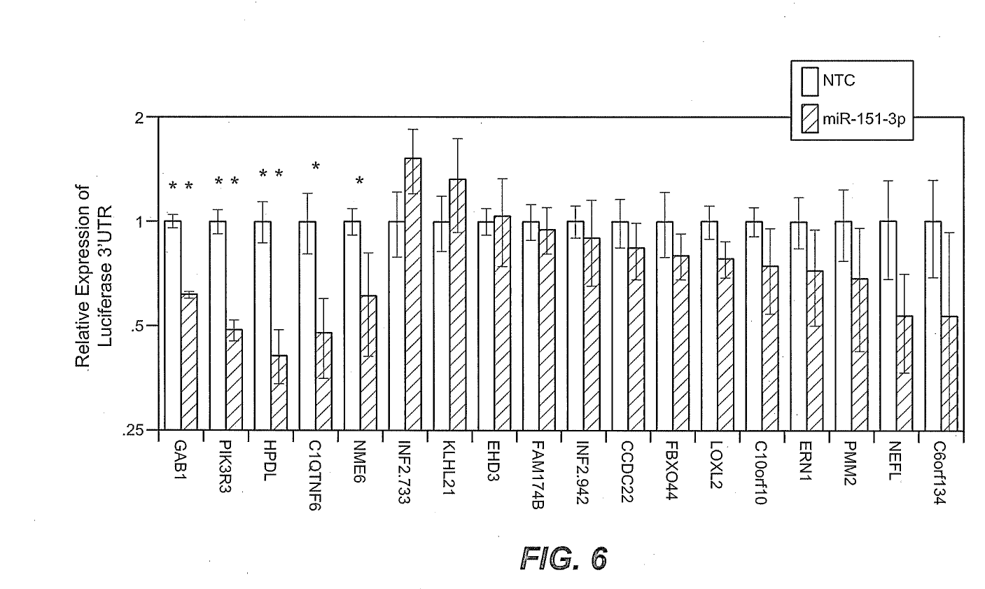

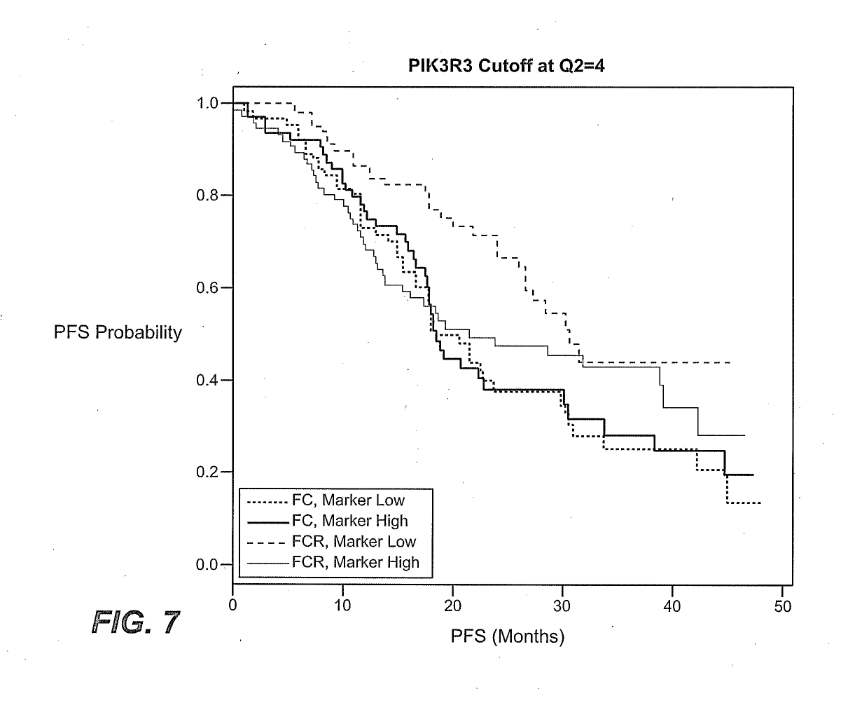

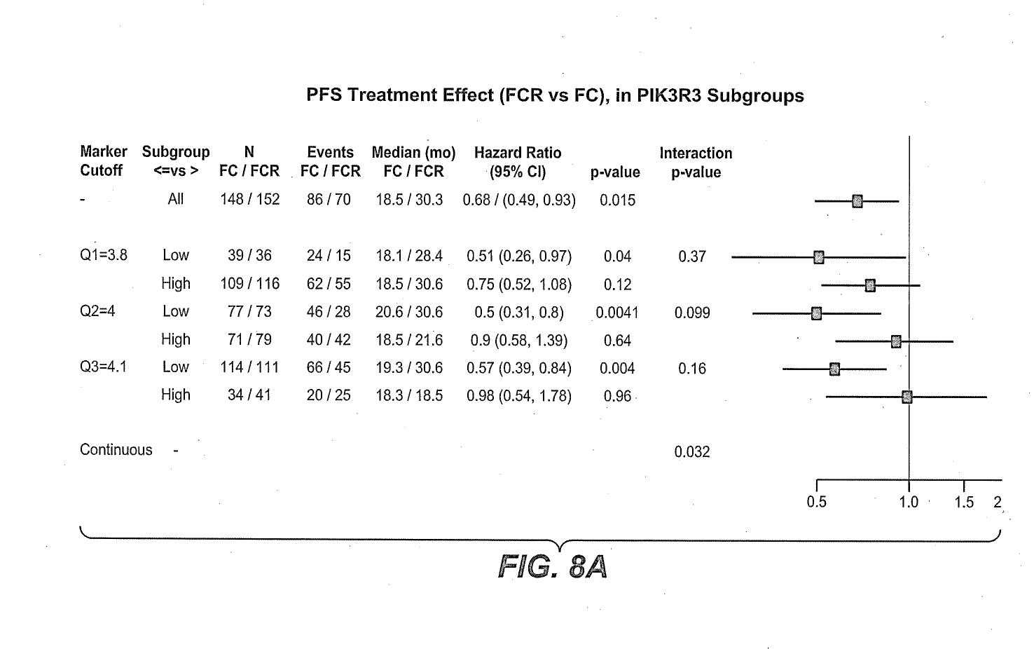

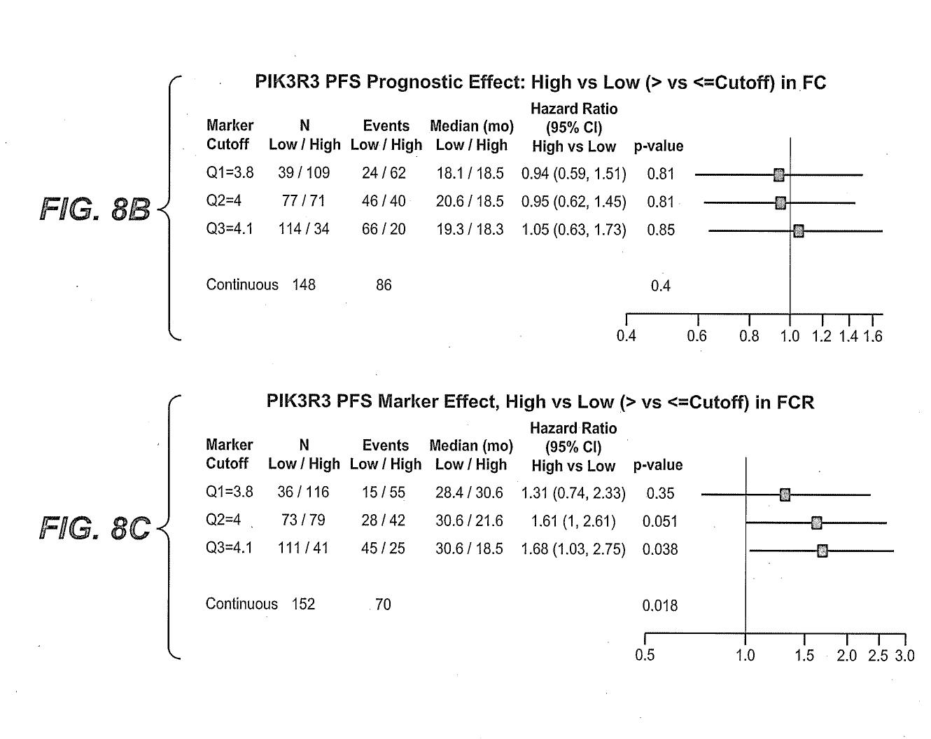

Target correlation coefficients were determined and only 23 predicted targets out of 1214 were negatively correlated with miRNA151 3p (PCC<−0.2 with q-value<0.01). To verify which of these predicted targets were likely to be bona fide targets, a mimic of miRNA151 3p or a scrambled control was expressed in HeLa cells and assessed for their ability to suppress expression of luciferase reporter constructs containing the 3′UTR of each gene after 24 hours (FIG. 6). Of the 23 predicted 3′UTRs, 18 could be subcloned into the appropriate vector. Notably, only 5 out of the 18 (28%) 3′UTRs showed a statistically significant reduction in luciferase activity upon introduction of miRNA151 3p. These results imply that miRNA151 3p is selectively regulating targets to mediate a specific biological response that could be modulating the response to FCR. Furthermore, the lower mRNA expression of one of these targets, PIK3R3, was positively associated with R-FC outcome (p=0.03, FIGS. 7 and 8A-C).

### Conclusions

The aim of this study was to discover biomarkers that predict the outcome of CLL patients treated with anti-CD20 antibody based therapy. The data herein reveals, for the first time, that elevated expression levels (above median) of miRNA151 3p and miRNA409 3p as well as elevated gene expression levels of PTK2 (above median) are associated with prolonged PFS in CLL patients treated with anti-CD20 based therapy. The findings from microarray platforms were validated by qRT-PCR. In addition, the predictive significance remained in multivariate analysis using factors known to influence the prognosis of the disease.

In conclusion, the data herein demonstrate the feasibility of predicting the outcome and benefit of CLL patients to anti-CD20 antibody based therapy prior to treatment. Diagnostic assessment of miRNAs 151 3p and 409 3p and mRNA expression of PTK2 serve as useful tools to select the most appropriate therapy for CLL patients. Furthermore, lower expression of PIK3R3, a confirmed target of miRNA151 3p, was positively associated with anti-CD20 antibody outcome.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method for treating a patient with chronic lymphocytic leukemia (CLL) comprising administering a therapeutically effective amount of a CLL medicament to the patient if the patient has been found to have an elevated amount of one or more biomarker selected from miRNA151 3p, miRNA409 3p, and PTK2.

2. The method of claim 1 wherein the CLL medicament induces FAK signaling or homotypic adhesion.

3. The method of claim 1 wherein the CLL medicament is a B-cell antagonist.

4. The method of claim 1 wherein the CLL medicament is an anti-CD20 antibody.

5. The method of claim 4 wherein the anti-CD20 antibody is a humanized, human, or chimeric anti-CD20 antibody.

6. The method of claim 4 wherein the anti-CD20 antibody is selected from the group consisting of rituximab, ofatumumab, GA101, SBI-087, veltuzumab, and AME-133.

7. The method of claim 6 wherein the anti-CD20 antibody is rituximab.

8. The method of any one of the preceding claims wherein the patient has greater progression free survival (PFS) relative to a patient who does not have an elevated amount of the biomarker.

9. The method of any one of the preceding claims further comprising administering chemotherapy to the patient.

10. The method of claim 9 wherein the chemotherapy comprises cyclophosphamide and fludarabine.

11. The method of claim 1 wherein the patient has an elevated amount of miRNA151 3p.

12. The method of claim 1 wherein the patient has an elevated amount of miRNA409 3p.

13. The method of claim 1 wherein the patient has an elevated amount of PTK2.

14. The method of claim 1 wherein the amount of the biomarker is evaluated by gene expression profiling.

15. The method of claim 14 wherein the gene expression profiling comprises polymerase chain reaction (PCR).

16. The method of claim 15 wherein the PCR comprises quantitative Real Time PCR (qRT-PCR).

17. The method of any one of the preceding claims comprising testing a sample from the patient for expression of the biomarker.

18. The method of claim 17 wherein the sample comprises Peripheral Blood Mononuclear Cells (PBMCs).

19. A method for treating a patient with chronic lymphocytic leukemia (CLL) comprising administering to the patient a therapeutically effective amount of a combination of rituximab, fludarabine and cyclophosphamide, if the patient has been found to have an elevated amount of one or more biomarker selected from miRNA151 3p, miRNA409 3p, and PTK2.

20. A method for treating a patient with chronic lymphocytic leukemia (CLL) comprising administering to the patient a therapeutically effective amount of CLL medicament other than rituximab, if the patient has been found to have a reduced amount of one or more biomarker selected from miRNA151 3p, miRNA409 3p, and PTK2.

21. A method for selecting a therapy for a patient with chronic lymphocytic leukemia (CLL) comprising determining expression of a biomarker selected from miRNA151 3p, miRNA409 3p, PTK2, and PI3K in a sample from the patient, and selecting a CLL medicament based on the level of expression of the biomarker.

22. The method of claim 21 wherein the patient is selected for treatment with a CLL medicament if the cancer sample expresses the biomarker at an elevated level.

23. The method of claim 21 wherein the CLL medicament induces FAK signaling or homotypic adhesion.

24. The method of claim 21 wherein the CLL medicament is a B-cell antagonist.

25. The method of claim 21 wherein the CLL medicament is an anti-CD20 antibody.

26. The method of claim 21 wherein the patient is selected for treatment with a CLL medicament other than rituximab if the cancer sample expresses the biomarker at a reduced level.

27. A diagnostic kit comprising one or more reagent for determining expression of a biomarker selected from miRNA151 3p, miRNA409 3p, PTK2, and PI3K, in a sample from a CLL patient.

28. The diagnostic kit of claim 27 further comprising instructions to use the kit to select a CLL medicament to treat the CLL patient.

29. The diagnostic kit of claim 27 or claim 28 wherein the one or more reagent comprises a pair of DNA primers and probe for detecting the biomarker.

30. An article of manufacture comprising, packaged together, a CLL medicament in a pharmaceutically acceptable carrier and a package insert indicating that the CLL medicament is for treating a patient with chronic lymphocytic leukemia (CLL) based on expression of one or more biomarker selected from miRNA151 3p, miRNA409 3p, PTK2, and PI3K.

31. A method for manufacturing an article of manufacture comprising combining in a package a pharmaceutical composition comprising a CLL medicament and a package insert indicating that the pharmaceutical composition is for treating a patient with chronic lymphocytic leukemia (CLL) based on expression of one or more biomarker selected from miRNA151 3p, miRNA409 3p, PTK2, and PI3K.

32. A method for advertising a CLL medicament comprising promoting, to a target audience, the use of the CLL medicament for treating a patient with chronic lymphocytic leukemia (CLL) based on expression of one or more biomarker selected from miRNA151 3p, miRNA409 3p, PTK2, and PI3K.

33. A method for treating a patient with chronic lymphocytic leukemia (CLL) comprising administering a therapeutically effective amount of a CLL medicament to the patient if the patient has been found to have reduced PI3K biomarker.

34. The method of claim 33 wherein the PI3K biomarker comprises PIK3R3.

35. A method for treating a patient with chronic lymphocytic leukemia (CLL) comprising administering to the patient a therapeutically effective amount of a combination of rituximab, fludarabine and cyclophosphamide, if the patient has been found to have reduced PI3K biomarker.

36. The method of claim 35 wherein the PI3K biomarker comprises PIK3R3.

37. A method for treating a patient with chronic lymphocytic leukemia (CLL) comprising administering to the patient a therapeutically effective amount of CLL medicament other than rituximab, if the patient has been found to have elevated PI3K biomarker.

38. The method of claim 37 wherein the PI3K biomarker comprises PIK3R3.
